(19) United States
(12) Patent Application Publication
(10) Pub. No.: US 2018/0045744 A1
(43) Pub. Date: Feb. 15, 2018

(54) NORMALIZING MEASURED DRUG CONCENTRATIONS IN ORAL FLUIDS AND TESTING FOR POTENTIAL NON-COMPLIANCE WITH DRUG TREATMENT REGIMEN

(71) Applicant: Ameritox, LLC, Baltimore, MD (US)

(72) Inventors: Gregory L. Mcintire, Greensboro, NC (US); Ayodele Morris, Midland, TX (US); Oneka Cummings, Greensboro, NC (US); Jeffrey Enders, High Point, NC (US)

(21) Appl. No.: 15/555,397

(22) PCT Filed: Jan. 15, 2016
(86) PCT No.: PCT/US16/13651

§ 371 Date: Sep. 1, 2017

Related U.S. Application Data
(60) Provisional application No. 62/104,486, filed on Jan. 16, 2015.

(51) Int. Cl. G01N 33/94; G06F 19/00

(52) U.S. Cl. CPC G01N 33/9486; G01N 33/94; G06F 19/3456

(57) ABSTRACT

Methods for monitoring subject compliance with a prescribed treatment regimen are disclosed. In an embodiment, the method comprises measuring a drug level in oral fluid of a subject and normalizing the measured drug level as a function of one or more parameters associated with the subject. Embodiments of the methods Use patient derived parameters together with the prescribed dose to affect a transformed and normalized value that can be compared to a transformed and normalized standard distribution derived from a body of collected oral fluid test results.

### PRIORITY CLAIM

This application claims priority to U.S. provisional application 62/104,486 filed Jan. 16, 2015 the entirety of which is incorporated herein by reference.

### TECHNICAL FIELD

The present disclosure provides methods for detecting and/or quantifying a subject's drug use and/or methods of assessing potential non-compliance with a drug treatment regimen by, inter alia, testing an oral fluid sample from said subject.

### BACKGROUND

Although hydrocodone (e.g., Vicodin, etc.) stands as the most prescribed opioid in the United States, the opioid that is responsible for the most emergency department (ED) visits in the United States is oxycodone (OXYCONTIN®). According to the Drug Abuse Warning Network (DAWN), approximately 77,000 ED visits in 2007 were due to the nonmedical use of oxycodone. The 2007 National Survey on Drug Use and Health estimates that 4.3 million Americans will abuse OXYCONTIN® sometime during the course of their lifetime. Hydrocodone shares similar statistics. In 2011, hydrocodone was the opioid responsible for the second highest ED visits (82,480) behind oxycodone (151,218 ED visits), as reported by DAWN. The Drug Enforcement Agency believes hydrocodone to be the most abused and diverted opioid in the United States. It is relatively inexpensive compared to oxycodone, which fosters its popularity. Given the propensity for abuse of oxycodone- and hydrocodone-containing medications and high incidence of ED visits associated with abuse, monitoring patients for compliance while being prescribed a pain regimen is an important component of their care.

Because of known dependency risks, subjects on opioid therapy regimens are typically screened periodically to monitor compliance and efficacy of the prescribed therapy (Webster, 2013). Due to the limits of known screening techniques, however, subjects misusing the prescribed opioid often pass basic screening tests performed at a clinic and continue to receive the opioid. Furthermore, patients treated with opioids for the management of chronic pain also have been documented to under-report their use of medications. As a result, health care professionals often use external sources of information such as interviews with the subject's spouse and/or friends, review of the subject's medical records, input from prescription monitoring programs, and testing of biological samples (e.g., fluids) to detect misuse of drugs and non-compliance with the prescribed opioid regimen.

Known drug screening methods generally can detect the presence or absence of a drug in a sample. Samples of fluids are generally obtained from the subject, for example, urine, blood, or plasma. Such known screening methods generally do not, however, enable the health care professional reviewing the lab result to determine whether the subject is non-compliant with a prescribed drug regimen. Determining compliance or non-compliance with a prescribed drug regimen using oral fluid samples has not yet been achieved, partly because the concentrations of drugs in oral fluids are often small. In addition, the half-life of a given drug is generally substantially shorter in oral fluid compared to the half-life of the drug and/or its metabolites in urine.

There are issues with securing samples of each of these fluids (Substance Abuse and Mental Health Services Administration, 2012; Vindenes et al., 2011; Bosker and Huestis, 2011); for example, requiring a phlebotomist to take blood samples in a licensed facility and the necessity of a private (bathroom) space for the provision of urine not to mention the ease of adulterating urine samples to hide or otherwise misdirect the lab test results.

While drug concentrations can be discerned in and from oral fluids, the results are not always directly translatable to compliance. Normalized curves for a series of drugs have been published for urine drug samples (Couto, et al., 2011; Couto, et al, 2009) such that a physician can quickly compare the patient's results with normalized data from a patient population to help determine the likelihood that the patient is compliant. While some have criticized these works (McCloskey, et al. 2013, McCloskey and Stickle 2013), the curves do have utility in everyday medical practice. However, normalized urinary curves cannot be used to assess compliance based on drug or metabolite concentrations in other fluids, and normalized oral fluid curves are so far unavailable to clinicians. Methods of assessing the risk of a patient's non-compliance with a prescribed drug regimen using a fluid other than urine, blood or plasma are therefore needed.

### SUMMARY

In various embodiments, the present invention provides methods for determining (e.g., detecting or monitoring) a subject's compliance or potential non-compliance with a prescribed drug regimen. In an embodiment, the present disclosure provides a method of identifying a subject at risk of drug misuse. In some embodiments, the present disclosure provides a method of reducing the risk of drug misuse in a subject by reducing a prescribed daily dose of a drug for the subject or counseling the subject if the drug concentration in oral fluid of the subject falls above the upper confidence interval (e.g. 2 standard deviations above the population mean) or above the upper limit of the mathematically transformed and normalized concentration range for the daily dose of the drug. In still other embodiments, the invention provides a method of helping to identify the risk of drug misuse in a subject by counseling the subject if the drug concentration in oral fluid of the subject falls below the lower confidence interval or below the lower limit (e.g., 2 standard deviations below the population mean) of the mathematically transformed and normalized concentration range for the daily dose of the drug. These and other embodiments can comprise performing mathematical transformations and normalization to yield a normalized drug concentration determined from an oral fluid sample from a subject and comparing that mathematically transformed and normalized drug concentration to a distribution curve prepared from a body of known test subjects who were both prescribed the drug of interest and tested positive for the drug and/or metabolite in oral fluids.

Embodiments of the invention can identify samples in the lower and upper extremes of a mathematically transformed normal distribution relevant to that drug. For example, embodiments of the invention can identify samples in the lower 2.5% and the upper 2.5% extremes of the mathematically transformed and normalized distribution of a specific drug concentration in oral fluid. Furthermore, relative to known methods, embodiments of the invention can improve differentiation between compliance and non-compliance for patients providing oral fluid samples for testing.

In some embodiments, methods of the present disclosure use a body of collected test results from oral fluid samples for the drug or drug metabolite of interest to form a mathematically transformed and normalized database. As opposed to conventional (i.e. urine) standard curves where carefully controlled, relatively small data sets (i.e., prospective clinical trials), are used to construct “normal” curves for comparison to current drug testing results, the present method uses data obtained for the drug or metabolite of the drug of interest and the accompanying demographics and dose data to construct a mathematically transformed and normalized standard curve for oral fluid testing results regardless of dose, time of sample donation, time of dosing, and concurrent medications (if any). Thus, the samples used for this mathematically transformed and normalized standard curve may include samples from subjects that are fast or slow metabolizers, subjects with impaired kidney or liver function, subjects using drugs with overlapping metabolites on the same day, and/or subjects taking medication on an inconsistent schedule. However, this process does exclude samples without a discrete value for the drug concentration in question (i.e., >Upper Limit of Linearity (ULOL) or <Lower Limit of Quantitation (LOQ)), samples that might have been positive for the drug of interest but obtained from subjects that were not prescribed that drug, etc. This top-down approach to preparing a mathematically transformed and normalized standard curve for oral fluid derived samples provides a reliable comparison of mathematically transformed and normalized oral fluid derived drug concentrations to an overall population comprised of more than 50 data points, more preferably more than 200 data points, and most preferably more than 1000 data points.

In other embodiments, both primary and secondary metabolites are measured allowing the use of a ratio of metabolite 1 to metabolite 2 or vice versa. It is envisioned that metabolite 1 may be the parent drug originally dosed to the patient. In some embodiments, two or more drug metabolites (e.g., primary, secondary, and/or tertiary metabolites) are determined, a ratio of one metabolite to at least one other metabolite is calculated, and a risk of the subject's noncompliance is determined if the ratio falls outside confidence intervals or mathematically transformed and normalized range of that ratio for the daily dose of the drug. In some embodiments, one metabolite is the parent drug originally dosed to the patient. In some embodiments, the ratio is of one metabolite to the sum of all metabolites.

In some embodiments, the use of calculated blood volume is critical to the normalization of the mathematically transformed data. Unlike urine, wherein creatinine concentration is commonly used to establish the level of “hydration” of the subject and further to normalize data to that level of hydration, creatinine is not expressed in oral fluid. However, in some embodiments (e.g., wherein a concentration of a drug and/or its plasma resident metabolites observed in oral fluid is representative of the concentration in blood or plasma), the calculated blood volume (CBV) is used to normalize all the subjects to the same blood volume results in a “normalized” standard curve.

These and other embodiments of the present disclosure are disclosed in further detail herein below.

### BRIEF DESCRIPTION OF THE DRAWINGS

FIG. 1 shows a histogram of the Hydrocodone drug concentrations observed from a body of collected oral fluid test results used to generate the mathematically transformed and normalized standard curve for Hydrocodone from oral fluids.

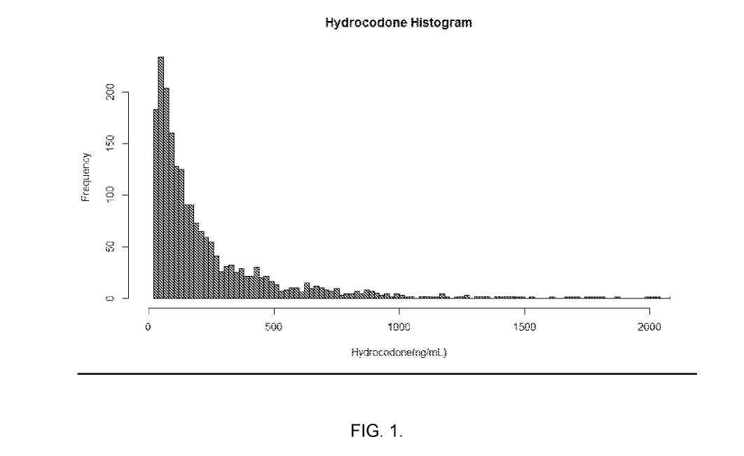

FIG. 2 shows the corresponding kernel density estimation plot derived from the data in FIG. 1. The kernel density estimation is a well-accepted mathematical tool that “smooths” continuous data (e.g., Histograms) such that mathematical curve fitting and modelling can be accomplished. While the variables used to construct the Kernel Density

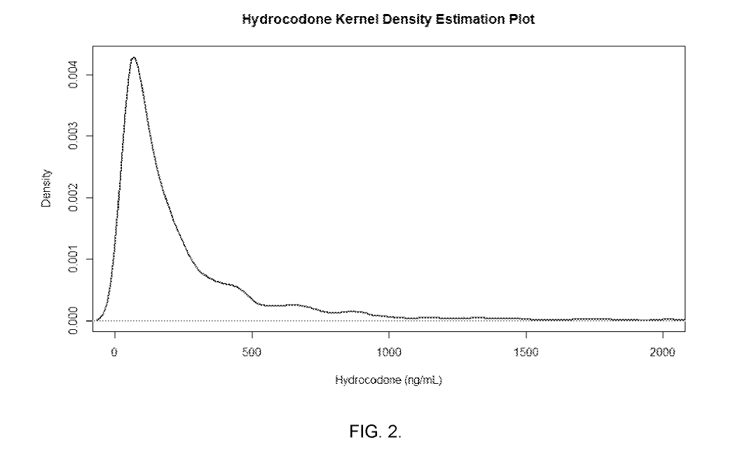

Estimation Plot can be subjective, the result for a continuous data set retains the mean value and closely reflects the variance of the original data set itself. The kernel density estimation plot is simply used to “clean up” the display for inspection (Parzen 1962).

FIG. 3 shows the impact of mathematically transforming the data presented in FIG. 1 using subject specific parameters or transformed variables arising from these parameters. Note, the raw data are transformed rather than the Kernel Density Estimated Data plot.

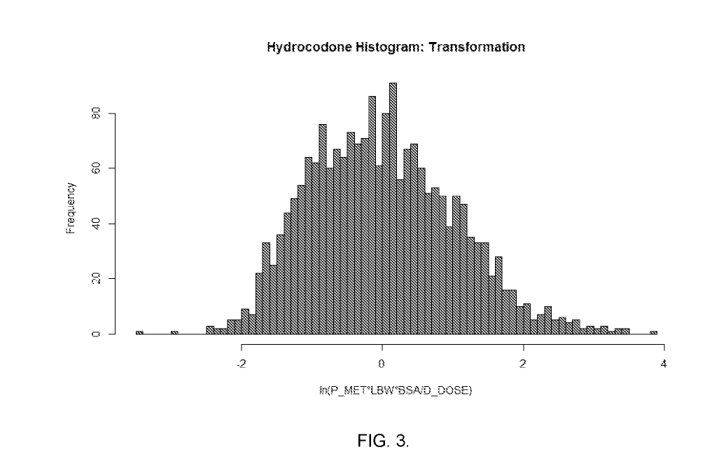

FIG. 4 shows the corresponding kernel density estimation plot derived from the mathematically transformed data presented in FIG. 3.

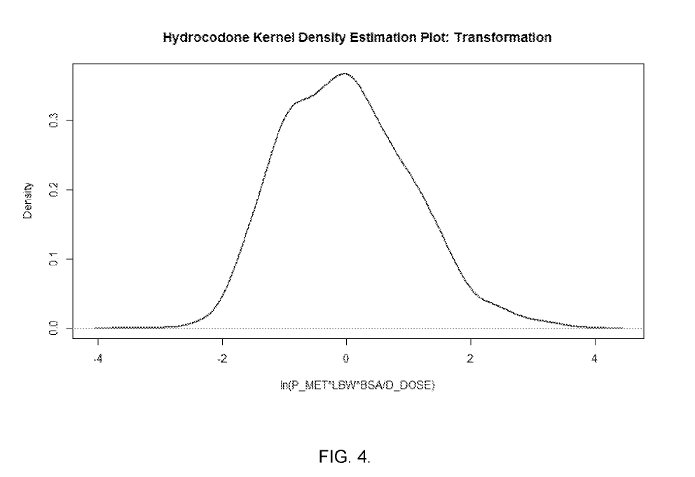

FIG. 5 shows the impact of normalizing these mathematically transformed data (FIG. 3) using calculated blood volume. Again, note, the transformed raw data were normalized rather than the kernel density estimated data. This model is described using Equation 1.

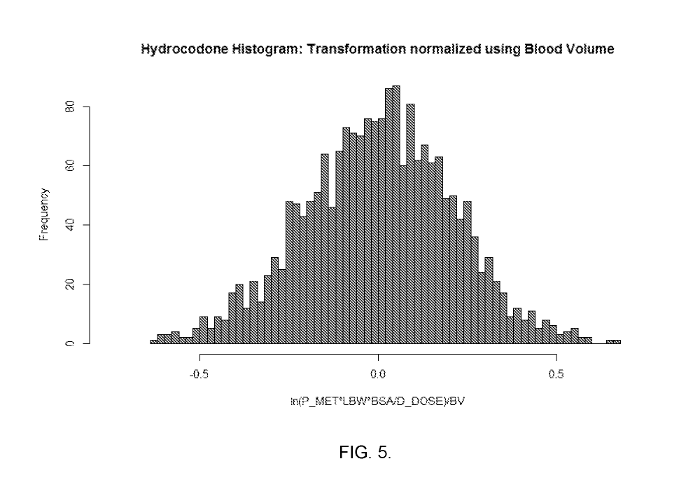

FIG. 6 shows the kernel density estimation plot derived from the normalized data shown in FIG. 5.

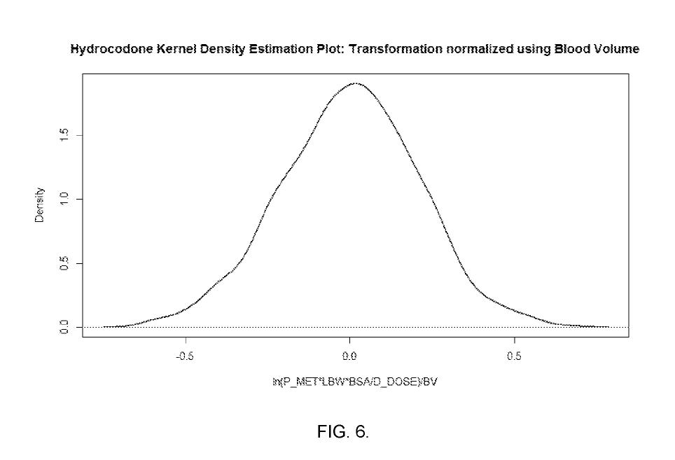

FIG. 7 shows a least squares minimized best fit Gaussian distribution curve derived from the transformed data from FIG. 1 (i.e., FIG. 3) and then normalized using calculated blood volume (FIG. 5). Again, it is important to note that these curves were derived from the raw data and not the kernel density estimated plot (data).

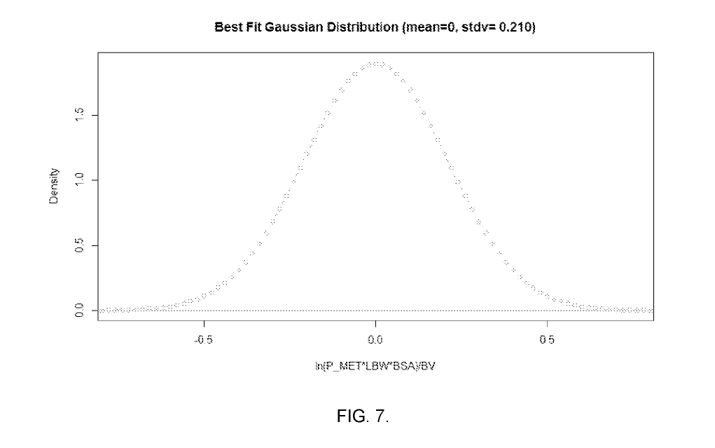

FIG. 8 shows a histogram of the Oxycodone drug concentrations observed from a body of oral fluid test results used to generate the mathematically transformed and normalized standard curve for Oxycodone from oral fluids.

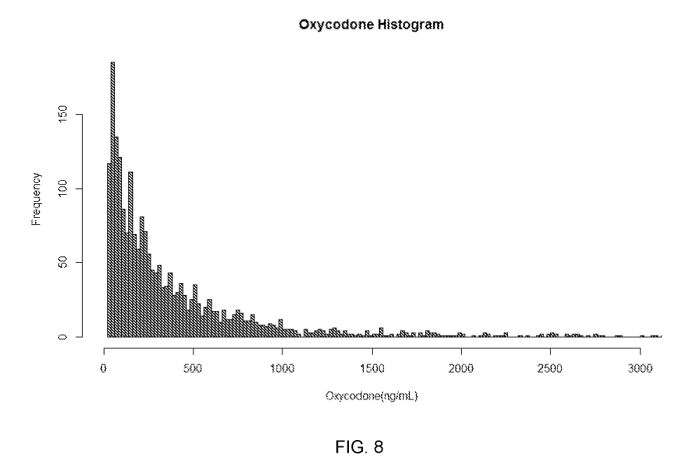

FIG. 9 shows the corresponding kernel density estimation plot derived from the data in FIG. 8. The kernel density estimation is a well accepted mathematical tool that “smooths” continuous data (e.g., Histograms) such that mathematical curve fitting and modelling can be accomplished. While the variables used to construct the Kernel Density Estimation Plot can be subjective, the result for a continuous data set retains the mean value and closely reflects the variance of the original data set itself. The kernel density estimation plot is simply used to “clean up” the display for inspection.

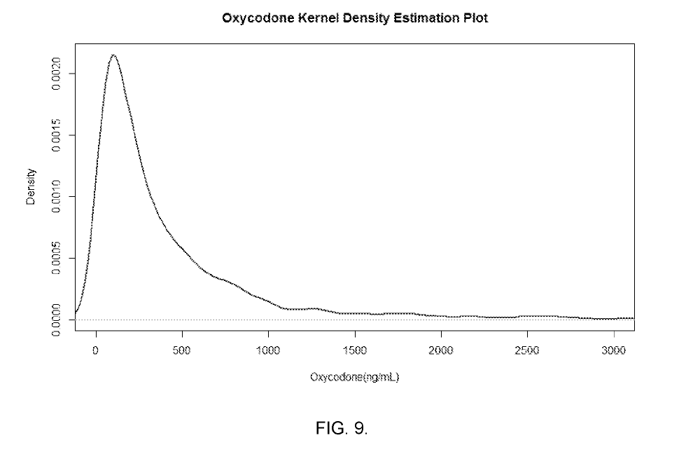

FIG. 10 shows the impact of mathematically transforming the data presented in FIG. 8.

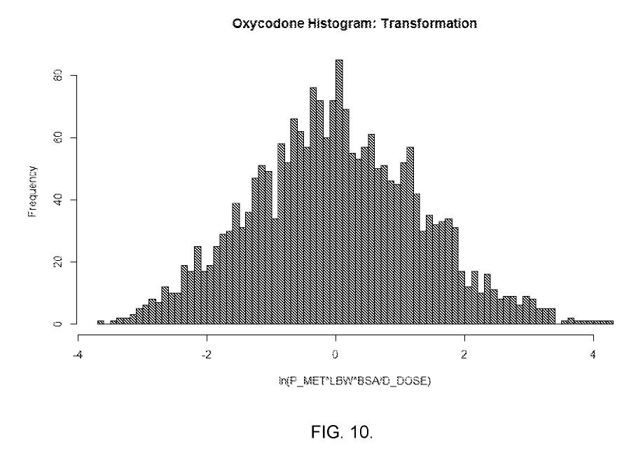

FIG. 11 shows the corresponding kernel density estimation plot derived from the mathematically transformed data presented in FIG. 10.

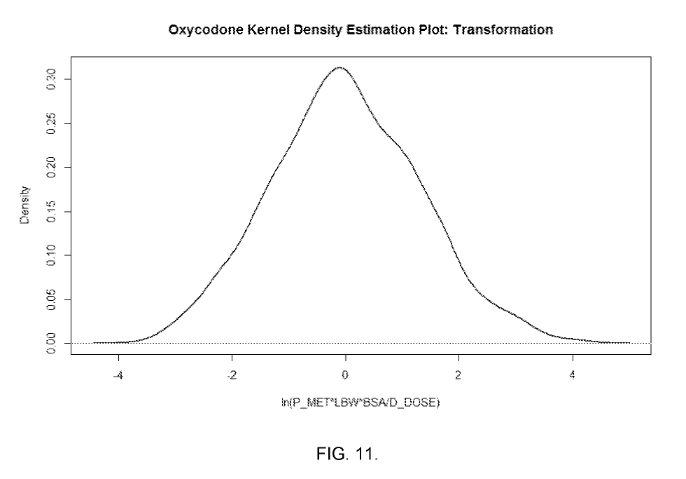

FIG. 12 shows the impact of normalizing these mathematically transformed data presented in FIG. 10 using calculated blood volume.

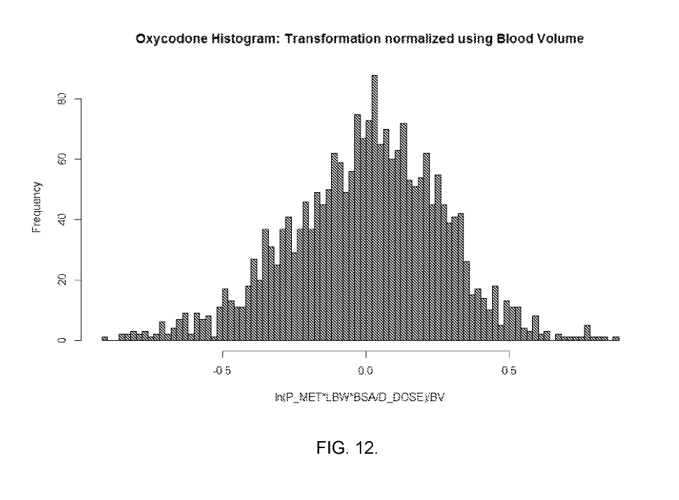

FIG. 13 shows the kernel density estimation plot derived from the normalized data shown in FIG. 13.

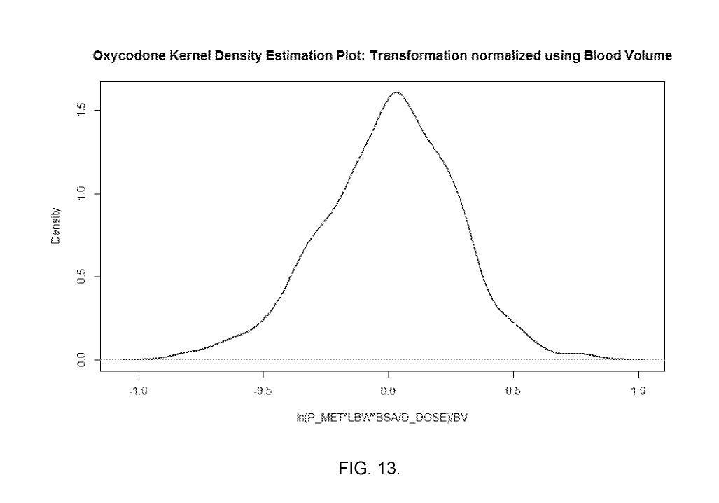

FIG. 14 shows a least squares minimized best fit Gaussian distribution derived from the transformed data from FIG. 10 and then normalized using calculated blood volume (FIG. 12).

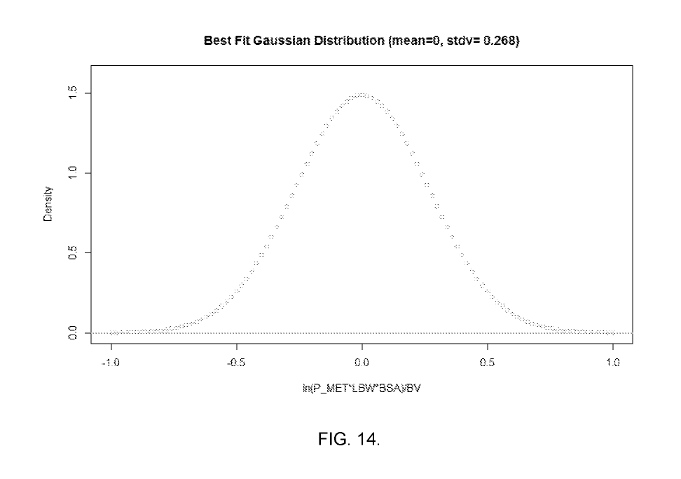

### DETAILED DESCRIPTION

While the present invention is capable of being embodied in various forms, the description below of several embodiments is made with the understanding that the present disclosure is to be considered as an exemplification of the invention, and is not intended to limit the invention to the specific embodiments illustrated. Headings are provided for convenience only and are not to be construed to limit the invention in any manner. Embodiments illustrated under any heading may be combined with embodiments illustrated under any other heading.

The use of numerical values in the various quantitative values specified in this application, unless expressly indicated otherwise, are stated as approximations as though the minimum and maximum values within the stated ranges were both preceded by the word “about.” Also, the disclosure of ranges is intended as a continuous range including every value between the minimum and maximum values recited as well as any ranges that can be formed by such values. Also disclosed herein are any and all ratios (and ranges of any such ratios) that can be formed by dividing a disclosed numeric value into any other disclosed numeric value. Accordingly, the skilled person will appreciate that many such ratios, ranges, and ranges of ratios can be unambiguously derived from the numerical values presented herein and in all instances such ratios, ranges, and ranges of ratios represent various embodiments of the present invention.

As used herein, the singular form of a word includes the plural, and vice versa, unless the context clearly dictates otherwise. Thus, the references “a”, “an”, and “the” are generally inclusive of the plurals of the respective terms. For example, reference to “an embodiment” or “a method” includes a plurality of such “embodiments” or “methods.” Similarly, the words “comprise”, “comprises”, and “comprising” are to be interpreted inclusively rather than exclusively. Likewise the terms “include”, “including” and “or” should all be construed to be inclusive, unless such a construction is clearly prohibited from the context. The terms “comprising” or “including” are intended to include embodiments encompassed by the terms “consisting essentially of” and “consisting of.” Similarly, the term “consisting essentially of” is intended to include embodiments encompassed by the term “consisting of”.

### Therapeutic Regimens

In one embodiment, the present invention provides a method to assist in detecting non-compliance or potential non-compliance with a prescribed drug regimen in a subject. The term “non-compliance” as used herein refers to any substantial deviation from a course of treatment that has been prescribed by a physician, nurse, nurse practitioner, physician's assistant, or other health care professional. A substantial deviation from a course of treatment may include any intentional or unintentional behavior by the subject that increases or decreases the amount, timing or frequency of drug ingested or otherwise administered (e.g., transdermal patch) compared to the prescribed therapy.

Non-limiting examples of substantial deviations from a course of treatment include: taking more of the drug than prescribed, taking less of the drug than prescribed, taking the drug more often than prescribed, taking the drug less often than prescribed, intentionally diverting at least a portion of the prescribed drug, unintentionally diverting at least a portion of the prescribed drug, etc. For example, a subject substantially deviates from a course of treatment by taking about 5% to about 1000% of the prescribed daily dose or prescribed drug regimen, for example about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 105%, about 110%, about 115%, about 120%, about 125%, about 150%, about 175%, about 200%, about 225%, about 250%, about 275%, about 300%, about 350%, about 400%, about 450%, about 500%, about 550%, about 600%, about 650%, about 700%, about 750%, about 800%, about 850%, about 900%, about 950%, or about 1000% of the prescribed drug regimen.

A subject may also substantially deviate from a course of treatment by taking about 5% to about 1000% more or less than the prescribed dose, for example about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 100%, about 125%, about 150%, about 175%, about 200%, about 225%, about 250%, about 275%, about 300%, about 350%, about 400%, about 450%, about 500%, about 550%, about 600%, about 650%, about 700%, about 750%, about 800%, about 850%, about 900%, about 950%, or about 1000% less than the prescribed dose. A subject may also substantially deviate from a course of treatment by, for example, taking the prescribed dose of a drug about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 100%, about 125%, about 150%, about 175%, about 200%, about 225%, about 250%, about 275%, about 300%, about 350%, about 400%, about 450%, about 500%, about 550%, about 600%, about 650%, about 700%, about 750%, about 800%, about 850%, about 900%, about 950%, or about 1000% more often or less often than specified in the course of treatment or prescribed in the drug regimen.

In another embodiment, a subject according to the present invention is prescribed a daily dose of a drug. The term “daily dose” or “prescribed daily dose” as used herein refers to any periodic administration of a drug to the subject over a given period of time, for example per hour, per day, per every other day, per week, per month, per year, etc. Preferably the daily dose or prescribed daily dose is the amount of the drug prescribed to a subject in any 24-hour period. While the drug may be administered according to any method known in the art including, for example, orally, intravenously, topically, transdermally, subcutaneously, sublingually, rectally, etc., for the purposes of this application, the test results must be derived from oral fluid samples. The prescribed daily dose of the drug may be approved by the Food & Drug Administration (“FDA”) for a given indication. In the alternative, a daily dose or a prescribed daily dose may be an unapproved or “off-label” use for a drug for which FDA has approved other indications. As a non-limiting example, FDA has approved oxycodone HCI controlled-release tablets (OXYCONTIN®) for use in the management of moderate to severe pain in 10 mg, 15 mg, 20 mg, 30 mg, 40 mg, 60 mg, 80 mg, 160 mg tablets. Any use of oxycodone HCI controlled-release tablets (OXYCONTIN®) other than to manage moderate to severe pain or at other than approved doses is an “off-label” use.

In various embodiments, methods according to the present invention involve the step of determining a prescribed dose of a drug. The term “determining a prescribed dose” as used herein refers to any method known to those in the art to ascertain, discover, deduce, or otherwise learn the dose of a particular drug that has been prescribed to the subject. Non-limiting examples include subject interview, consultation with the subject's medical history, consultation with another health care professional familiar with the subject, consultation with a medical record associated with the subject, etc.

The term “drug” as used herein refers to an active pharmaceutical ingredient (“API”) and its metabolites, decomposition products, enantiomers, diastereomers, derivatives, etc.

In some embodiments, the drug is an opioid. The term “opioid” as used herein refers to any natural, endogenous, synthetic, or semi-synthetic compound that binds to opioid receptors. Non-limiting examples of opioids include: codeine, morphine, thebaine, oripavine, diacetylmorphine, dihydrocodeine, hydrocodone, hydromorphone, nicomorphone, oxycodone, oxymorphone, fentanyl, alphamethylfentanyl, alfentanil, sufentanil, remifentanil, carfentanyl, ohmefentanyl, pethidine, keobem idone, desmethylprodine, (“MPPP”), allylprodine, prodine, 4-phenyl-1-(2-phenylethyl)piperidin-4-yl acetate (“PEPAP”), propoxyphene, dextropropoxyphene, dextromoramide, bezitramide, piritramide, methadone, dipipanone, levomathadyl acetate (“LAAM”), difenoxin, diphenoxylate, loperamide, dezocine, pentazocine, phenazocine, buprenorphine, dihydroetorphine, etorphine, butorphanol, nalbuphine, levorphanol, levomethorphan, lefetamine, meptazinol, tilidine, tramadol, tapentadol, nalmefene, naloxone, naltrexone, methadone, derivatives thereof, metabolites thereof, prodrugs thereof, controlled-release formulations thereof, extended-release formulations thereof, sustained-release formulations thereof, and combinations of the foregoing.

In an embodiment, a method according to the present invention confirms a subject's non-adherence to a chronic opioid therapy (“COT”). The term “chronic opioid therapy” as used herein refers to any short-term, mid-term, or long-term treatment regimen comprising at least one opioid. As a non-limiting example, a subject suffering chronic pain may ingest a daily dose of oxycodone to relieve persistent pain resulting from trauma, chronic conditions, etc. COT is generally prescribed to a subject in need of such therapy; subjects on COT are typically monitored periodically by a health care professional for addiction, tolerance, or other common outcomes associated with COT. In one embodiment, a method according to the present invention assists a health care professional in confirming a subject's adherence or non-adherence to a COT regimen.

Subjects on COT sometimes develop an addiction to the prescribed opioid. Studies have shown that a subject on COT is more likely to develop an addiction to a prescribed opioid when he or she has a history of aberrant drug-related behavior, or is at high risk of aberrant drug-related behavior. The term “aberrant drug-related behavior” as used herein refers to any behavioral, genetic, social, or other characteristic of the subject that tends to predispose the subject to development of an addiction for an opioid.

Non-limiting examples of such risk factors include a history of drug abuse, a history of opioid abuse, a history of non-opioid drug abuse, a history of alcohol abuse, a history of substance abuse, a history of prescription drug abuse, a low tolerance to pain, a high rate of opioid metabolism, a history of purposeful over-sedation, negative mood changes, intoxicated appearance, an increased frequency of appearing unkempt or impaired, a history of auto or other accidents, frequent early renewals of prescription medications, a history of or attempts to increasing dose without authorization, reports of lost or stolen medications, a history of contemporaneously obtaining prescriptions from more than one doctor, a history of altering the route of administering drugs, a history of using pain relief medications in response to stressful situations, insistence on certain medications, a history of contact with street drug culture, a history of alcohol abuse, a history of illicit drug abuse, a history of hoarding or stockpiling medications, a history of police arrest, instances of abuse or violence, a history of visiting health care professionals without an appointment, a history of consuming medications in excess of the prescribed dose, multiple drug allergies and/or intolerances, frequent office calls and visits, a genetic mutation that up-regulates or down-regulates production of drug metabolizing enzymes, a reduced-function CYP2D6 allele, and/or a non-functional CYP2D6 allele.

In an embodiment, the present invention assists a health care professional in assessing a risk that a subject is misusing a prescribed drug. For example, based on the comparison of the mathematically transformed and normalized datum to the same mathematically transformed and normalized standard distribution performed in embodiments of the present invention, a healthcare worker can intervene (e.g. via counseling, modifying the subject's regiment/dose, etc.) in the subject's misuse on the basis of the risk assessment.

### Sample Measurement

Methods according to the present invention may be used to determine the comparison of a mathematically transformed and normalized datum to a similarly transformed and normalized standard distribution of a wide variety of drugs in oral fluids of a subject. When the fluid analyzed is oral fluid, for example, methods according to the present invention may be used to determine the comparison of any drug that can be measured in an oral fluid sample to a like standard distribution.

In some embodiments, the amount of a drug in a subject is determined by analyzing a fluid of the subject. The term “fluid” as used herein refers to oral fluid and any liquid or pseudo-liquid obtained from the oral cavity of the subject. Non-limiting examples include saliva, mucus, and the like. In an embodiment, the fluid is oral fluid.

Determining the amount of a drug in oral fluid of the subject may be accomplished by use of any method known to those skilled in the art. Non-limiting examples for determining the amount of a drug in fluid of a subject include fluorescence polarization immunoassay (“FPIA,” Abbott Diagnostics), mass spectrometry (MS), gas chromatography-mass spectrometry (GC-MS-MS), liquid chromatography-mass spectrometry (LC-MS-MS), and the like. In one embodiment, LC-MS-MS methods known to those skilled in the art are used to determine a raw level, amount or concentration of a drug in oral fluid of the subject. In one embodiment, a raw level or concentration of a drug in oral fluid of a subject is measured and reported as a ratio, percent, or in relationship to the amount of fluid. The amount of fluid may be expressed as a unit volume, for example, in L, mL, μL, pL, ounce, etc. In one embodiment, the raw amount of a drug in oral fluid of a subject may be expressed as an absolute level or value, for example, in g, mg, pg, ng, pg, etc.

In some embodiments, the level, concentration or amount of a drug determined in oral fluid of a subject is transformed and normalized. The term “normalized” as used herein refers to a level or concentration of a drug that has been modified to correct for one or more parameters associated with the subject. Non-limiting examples of parameters include: sample fluid pH, sample fluid specific gravity, sample fluid salt concentration, subject height, subject weight, subject age, subject body mass index, subject gender, subject lean body mass, subject calculated blood volume, subject total body water volume, and subject body surface area, subject prescribed drug dosage. Part of the normalization process requires adjusting the concentration of the drug and other parameters associated with the subject so that they share a common sale (ensuring that all units are consistent). Parameters may be measured by any means known in the art. For example, sample fluid pH may be measured using a pH meter, litmus paper, test strips, etc. In some embodiments, the level, concentration or amount of a drug in oral fluid is normalized and then transformed as a function of the natural logarithm of the parametrically normalized sample concentration.

In an embodiment, the transformed and normalized drug concentration is normalized using subject height, subject weight, subject gender, subject body mass index, subject lean body weight, subject body surface area, subject prescribed drug dosage, and subject calculated blood volume. In a related embodiment, the transformed and normalized drug concentration is determined without using subject calculated blood volume. In another related embodiment, the transformed and normalized drug concentration is determined from the primary metabolite concentration using parameters consisting of subject height, subject weight, subject gender, subject body mass index, subject lean body weight, subject prescribed drug dosage, and subject calculated blood volume In yet another related embodiment, the transformed and normalized drug concentration is determined from the primary metabolite concentration and the secondary metabolite concentration using parameters consisting of primary metabolite concentration, secondary metabolite concentration, subject height, subject weight, subject gender, subject body mass index, subject lean body weight, subject body surface area, subject prescribed drug dosage, and subject calculated blood volume. The parent drug is also referred to as the primary metabolite in some embodiments, for example when the parent drug is metabolized sufficiently slowly that it is directly measurable in a patient sample.

In one embodiment, a raw level or concentration of a drug in urine of a subject is measured and reported as a ratio, percent, or in relationship to the amount of fluid. In such embodiments, the normalized drug ratio concentration may be determined using parameters comprising subject age, subject weight, subject gender and creatinine concentration. In a related embodiment, the normalized drug concentration is determined without using sample fluid pH or subject lean body mass or subject calculated blood volume but rather subject total body water volume. In another related embodiment, the normalized drug concentration is determined from the ratio of the primary metabolites concentrations using parameters consisting of subject age, subject weight, subject gender and sample fluid creatinine. In yet another related embodiment, the normalized drug concentration ratio is determined from the primary metabolite concentration and the secondary metabolite concentration using a ratio of primary metabolite to secondary metabolite or vice versa with parameters consisting of primary metabolite concentration, secondary metabolite concentration, subject age, subject weight, subject gender and sample creatinine concentration. The primary metabolite can be the parent drug itself instead of an actual metabolite in the true sense.

In an embodiment, the raw drug concentration measured in oral fluid of the subject is transformed and normalized as a function of subject height, subject weight, subject gender, subject body mass index, subject lean body weight, subject body surface area, subject prescribed drug dosage, and subject calculated blood volume. (hereafter “Equation 1”):

$${NORM}_{D \_ CONC} = \frac{ln ( \frac{P\_MET * LBW * BSA}{D\_DOSE} )}{CBV} + ADJ\_A \qquad ( 1 )$$

Where In is the natural log, P_MET is the concentration of the primary metabolite also referred to as the parent drug in kg/L; LBW is the lean body weight of the subject in kg; BSA is the body surface area of the subject in meters squared; D_DOSE is the subject prescribed daily drug dosage in kg; and CBV is the calculated blood volume in liters. ADJ_A is a parameter that is derived from and specific to a given data set. It “moves” the mean of the transformed and normalized data set to a value of “0” such that variation from the mean in “standard deviation units” is readily observed. For example, from the data sets for hydrocodone and oxycodone, ADJ_A =0.148 for hydrocodone and 0.152 for oxycodone. The +1 standard deviations of the model described in Equation 1 applied to the data sets herein is 0.210 for hydrocodone and 0.268 for oxycodone.

In an embodiment, if the primary metabolite concentration is measured as zero or below the limit of detection of the method for a patient prescribed the drug, Equation 1 cannot be utilized and said patient will be deemed as potentially non-compliant. Alternatively, in the case where the primary metabolite concentration is less than the analytical method limit of quantitation (LOQ), a predetermined minimum value can be used to describe the data. As a non-limiting example, the predetermined minimum primary metabolite value and/or the predetermined minimum secondary metabolite value for use in Equation 1 can be 30 ng/mL or 3×10⁻⁸ kg/L. As another non-limiting example, the predetermined minimum primary metabolite value and/or the predetermined minimum secondary metabolite value for use in Equation 1 can be 10 ng/mL or 1×10⁻⁸ kg/L. As yet another non-limiting example, the predetermined minimum primary metabolite value and/or the predetermined minimum secondary metabolite value for use in Equation 1 can be 1 ng/mL or 1×10⁻⁹ kg/L. As a non-limiting example, the predetermined minimum primary metabolite value and/or the predetermined minimum secondary metabolite value for use in Equation 1 can be as low as the method of detection is capable of quantitating the value (e.g., Limit of Quantitation) which is dependent upon instrumentation and sample preparation as is well known by those skilled in the art.

In an embodiment, if the primary metabolite concentration is measured as zero, the primary metabolite concentration is used in Equation 1 as a different value, such as, for example, a predetermined minimum primary metabolite value for use in Equation 1. Additionally or alternatively, if the secondary metabolite concentration is measured as zero, the secondary metabolite concentration is used in Equation 1 as a different value, such as, for example, a predetermined minimum secondary metabolite value for use in Equation 1. As a non-limiting example, the predetermined minimum primary metabolite value and/or the predetermined minimum secondary metabolite value for use in Equation 1 can be 15 ng/mL. As a non-limiting example, the predetermined minimum primary metabolite value and/or the predetermined minimum secondary metabolite value for use in Equation 1 can be 1 ng/mL. As a non-limiting example, the predetermined minimum primary metabolite value and/or the predetermined minimum secondary metabolite value for use in Equation 1 can be 0.1 ng/mL

In a related embodiment, for a subject prescribed hydrocodone, a transformed and normalized drug level is determined from a raw level of the primary metabolite or the secondary metabolite as a function of subject height, subject weight, subject gender, subject body mass index, subject lean body weight, subject body surface area, subject prescribed drug dosage, and subject calculated blood volume, according to Equation 1. In a related embodiment, hydrocodone is the only opioid prescribed to the subject.

In a related embodiment, for a subject prescribed controlled-release oxycodone (OXYCONTIN®), a transformed and normalized drug level is determined from a raw level of the primary metabolite and the secondary metabolite as a function of subject height, subject weight, subject gender, subject body mass index, subject lean body weight, subject body surface area, subject prescribed drug dosage, and subject calculated blood volume, according to Equation 1. In a related embodiment, controlled-release oxycodone (OXYCONTIN®) is the only opioid prescribed to the subject.

In a related embodiment, for a subject prescribed oxycodone, a transformed and normalized drug level is determined from a raw level of the primary metabolite and the secondary metabolite as a function of subject height, subject weight, subject gender, subject body mass index, subject lean body weight, subject body surface area, subject prescribed drug dosage, and subject calculated blood volume, according to Equation 1. In a related embodiment, oxycodone is the only opioid prescribed to the subject.

In an embodiment, the raw drug concentration measured in oral fluid of the subject is transformed and normalized as a function of subject height, subject weight, subject gender, subject body mass index, subject lean body weight, subject prescribed drug dosage, and subject calculated blood volume. (hereafter “Equation 2”):

$${NORM}_{D \_ CONC} = \frac{ln ( \frac{P\_MET * LBW}{D\_DOSE} )}{CBV} + ADJ\_B \qquad ( 2 )$$

Where In is the natural log, P_MET is the concentration of the primary metabolite also referred to as the parent drug in kg/L; LBW is the lean body weight of the subject in kg; D_DOSE is the subject prescribed drug dosage in kg; and CBV is the calculated blood volume in liters. ADJ_B is a parameter that is derived from and specific to a given data set. It “moves” the mean of the transformed and normalized data set to a value of “0” such that variation from the mean in “standard deviation units” is readily observed. For example, from the data sets for hydrocodone and oxycodone herein, ADJ_B=0.276 for hydrocodone and 0.279 for oxycodone. The +1 standard deviation of the model described in Equation 2 for these data sets is 0.211 for hydrocodone and 0.269 for oxycodone.

In an embodiment, if the primary metabolite concentration is measured as zero or below the limit of detection of the method for a patient prescribed the drug, Equation 2 cannot be utilized and said patient will be deemed as potentially non-compliant. Alternatively, in the case where the primary metabolite concentration is less than the analytical method limit of quantitation (LOQ), a predetermined minimum value can be used to describe the data. As a non-limiting example, the predetermined minimum primary metabolite value and/or the predetermined minimum secondary metabolite value for use in Equation 2 can be 30 ng/mL or 3×10⁻⁸ kg/L. As another non-limiting example, the predetermined minimum primary metabolite value and/or the predetermined minimum secondary metabolite value for use in Equation 2 can be 10 ng/mL or 1×10⁻⁸ kg/L. As yet another non-limiting example, the predetermined minimum primary metabolite value and/or the predetermined minimum secondary metabolite value for use in Equation 2 can be 1 ng/mL or 1×10⁻⁹ kg/L. As a non-limiting example, the predetermined minimum primary metabolite value and/or the predetermined minimum secondary metabolite value for use in Equation 2 can be as low as the method of detection is capable of quantitating the value (e.g., Limit of Quantitation) which is dependent upon instrumentation and sample preparation as is well known by those skilled in the art.

In an embodiment, if the primary metabolite concentration is measured as zero, the primary metabolite concentration is used in Equation 2 as a different value, such as, for example, a predetermined minimum primary metabolite value for use in Equation 2. Additionally or alternatively, if the secondary metabolite concentration is measured as zero, the secondary metabolite concentration is used in Equation 2 as a different value, such as, for example, a predetermined minimum secondary metabolite value for use in Equation 2. As a non-limiting example, the predetermined minimum primary metabolite value and/or the predetermined minimum secondary metabolite value for use in Equation 2 can be 15 ng/mL. As a non-limiting example, the predetermined minimum primary metabolite value and/or the predetermined minimum secondary metabolite value for use in Equation 2 can be 1 ng/mL. As a non-limiting example, the predetermined minimum primary metabolite value and/or the predetermined minimum secondary metabolite value for use in Equation 2 can be 0.1 ng/mL

In a related embodiment, for a subject prescribed hydrocodone, a transformed and normalized drug level is determined from a raw level of the primary metabolite and the secondary metabolite as a function of subject height, subject weight, subject gender, subject body mass index, subject lean body weight, subject prescribed drug dosage, and subject calculated blood volume, according to Equation 2. In a related embodiment, hydrocodone is the only opioid prescribed to the subject.

In a related embodiment, for a subject prescribed controlled-release oxycodone (OXYCONTIN®), a transformed and normalized drug level is determined from a raw level of the primary metabolite and the secondary metabolite as a function of subject height, subject weight, subject gender, subject body mass index, subject lean body weight, subject body surface area, subject prescribed drug dosage, and subject calculated blood volume, according to Equation 2. In a related embodiment, controlled-release oxycodone (OXYCONTIN®) is the only opioid prescribed to the subject.

In a related embodiment, for a subject prescribed oxycodone, a transformed and normalized drug level is determined from a raw level of the primary metabolite and the secondary metabolite as a function of subject height, subject weight, subject gender, subject body mass index, subject lean body weight, subject body surface area, subject prescribed drug dosage, and subject calculated blood volume, according to Equation 2. In a related embodiment, oxycodone is the only opioid prescribed to the subject.

In an embodiment, the raw drug concentration measured in oral fluid of the subject is normalized as a function of subject height, subject weight, subject gender, subject body mass index, subject lean body weight, subject body surface area, and subject calculated blood volume. (hereafter “Equation 3”):

$${NORM}_{D \_ CONC} = \frac{ln ( P\_MET * LBW * BSA )}{CBV} + ADJ\_C \qquad ( 3 )$$

Where In is the natural log, P_MET is the concentration of the primary metabolite also referred to as the parent drug in kg/L; LBW is the lean body weight of the subject in kg; BSA is the body surface area of the subject in meters squared; and CBV is the calculated blood volume in L. ADJ_C is a parameter that is derived from and specific to a given data set. It “moves” the mean of the transformed and normalized data set to a value of “0” such that variation from the mean in “standard deviation units” is readily observed. For example, from the data sets for hydrocodone and oxycodone used herein, ADJ_C=2.113 for hydrocodone and 2.051 for oxycodone. The +1 standard deviation of the model described in Equation 3 for the data sets used herein is 0.584 for hydrocodone and 0.633 for oxycodone.

As aforementioned in other embodiments, the “adjustment parameters” ADJ_A, ADJ_B, and ADJ_C are derived from and specific to given data sets. These parameters are necessary to “move” the mean of the transformed and normalized data sets to a value of “0” such that variation from the mean in “standard deviation units” is readily observed. These “adjustment parameters” are summarized in Table 1 for Equation 1, Equation 2, and Equation 3 corresponding to models defined for both Hydrocodone and Oxycodone.

TABLE 1
Summary of the Adjustment Parameters used to move
the population means for the models described
in Equation 1, Equation 2, and Equation 3.

| | Hydrocodone | | Oxycodone | |
|---|---|---|---|---|
| | Adjustment | Standard | Adjustment | Standard |
| Equation Number | Parameter | Deviation | Parameter | Deviation |
| Equation 1: ADJ_A | 0.148 | 0.210 | 0.152 | 0.268 |
| Equation 2: ADJ_B | 0.276 | 0.211 | 0.279 | 0.269 |
| Equation 3: ADJ_C | 2.113 | 0.633 | 2.051 | 0.584 |

In an embodiment, if the primary metabolite concentration is measured as zero or below the limit of detection of the method for a patient prescribed the drug, Equation 3 cannot be utilized and said patient will be deemed as potentially non-compliant. Alternatively, in the case where the primary metabolite concentration is less than the analytical method limit of quantitation (LOQ), a predetermined minimum value can be used to describe the data. As a non-limiting example, the predetermined minimum primary metabolite value and/or the predetermined minimum secondary metabolite value for use in Equation 3 can be 30 ng/mL or 3×10⁻⁸ kg/L. As another non-limiting example, the predetermined minimum primary metabolite value and/or the predetermined minimum secondary metabolite value for use in Equation 3 can be 10 ng/mL or 1×10⁻⁸ kg/L. As yet another non-limiting example, the predetermined minimum primary metabolite value and/or the predetermined minimum secondary metabolite value for use in Equation 3 can be 1 ng/mL or 1×10⁻⁹ kg/L. As a non-limiting example, the predetermined minimum primary metabolite value and/or the predetermined minimum secondary metabolite value for use in Equation 3 can be as low as the method of detection is capable of quantitating the value (e.g., Limit of Quantitation) which is dependent upon instrumentation and sample preparation as is well known by those skilled in the art.

In an embodiment, if the primary metabolite concentration is measured as zero, the primary metabolite concentration is used in Equation 3 as a different value, such as, for example, a predetermined minimum primary metabolite value for use in Equation 3. Additionally or alternatively, if the secondary metabolite concentration is measured as zero, the secondary metabolite concentration is used in Equation 3 as a different value, such as, for example, a predetermined minimum secondary metabolite value for use in Equation 3. As a non-limiting example, the predetermined minimum primary metabolite value and/or the predetermined minimum secondary metabolite value for use in Equation 3 can be 15 ng/mL. As a non-limiting example, the predetermined minimum primary metabolite value and/or the predetermined minimum secondary metabolite value for use in Equation 3 can be 1 ng/mL. As a non-limiting example, the predetermined minimum primary metabolite value and/or the predetermined minimum secondary metabolite value for use in Equation 3 can be 0.1 ng/mL

In a related embodiment, for a subject prescribed hydrocodone, a transformed and normalized drug level is determined from a raw level of the primary metabolite and the secondary metabolite as a function of subject height, subject weight, subject gender, subject body mass index, subject lean body weight, subject prescribed drug dosage, and subject calculated blood volume, according to Equation 3. In a related embodiment, hydrocodone is the only opioid prescribed to the subject.

In a related embodiment, for a subject prescribed controlled-release oxycodone (OXYCONTIN®), a transformed and normalized drug level is determined from a raw level of the primary metabolite and the secondary metabolite as a function of subject height, subject weight, subject gender, subject body mass index, subject lean body weight, subject body surface area, subject prescribed drug dosage, and subject calculated blood volume, according to Equation 3. In a related embodiment, controlled-release oxycodone (OXYCONTIN®) is the only opioid prescribed to the subject.

In a related embodiment, for a subject prescribed oxycodone, a transformed and normalized drug level is determined from a raw level of the primary metabolite and the secondary metabolite as a function of subject height, subject weight, subject gender, subject body mass index, subject lean body weight, subject body surface area, subject prescribed drug dosage, and subject calculated blood volume, according to Equation 3. In a related embodiment, oxycodone is the only opioid prescribed to the subject.

In an embodiment, the concentration or level of drug in oral fluid of the subject is a steady state concentration or level. The term “steady state” as used herein refers to an equilibrium level or concentration of a drug obtained at the end of a certain number of administrations (e.g. 1 to about 5). Steady state is achieved when the concentration or level of the drug will remain substantially constant if the dose and the frequency of administrations remain substantially constant.

The parameters considered in the normalization Equation 1, Equation 2, and Equation 3 include subject height, subject weight, subject gender, subject body mass index, subject lean body weight, subject body surface area, subject prescribed drug dosage, and subject calculated blood volume. All of these parameters were all utilized in some modified or direct form in these mathematical transformed and normalized data points.

The lean body weight (LBW)—measured in kilograms—parameter accounts for the sum of everything in the human body with the exception of fat including but not limited to bones, muscles, and organs. The LBW is calculated using the James Formula (Absalom et al.,2009):

$$LBW ( kg ) = fact\_a * weight ( kg ) - fact\_b * {( \frac{weight ( kg )}{100 * height ( m )} )}^{2} \qquad ( 4 )$$

Where fact_a equals 1.1 for Men and 1.07 for Women and fact_b equals 128 for Men and 148 for women. Weight is the subject weight measured in kg and height is the subject height in m.

The body surface area (BSA)—measured in meters squared—parameter is the calculated surface area of the human body or the subject in this specific case. This accounts for subject BSA which is considered a better indicator of metabolic mass than the raw weight of the subject. The BSA is calculated using the Mosteller Method (Mosteller, 1987):

$$BSA ( {m}^{2} ) = \sqrt{( \frac{height ( cm ) * weight ( kg )}{3600} )} \qquad ( 5 )$$

Weight is the subject weight measured in kg and height is the subject height measured in cm.

The calculated blood volume (CBV)—measured in liters—parameter accounts for the volume of blood (both red blood cells and plasma) in the circulatory system of a subject. The CBV of each subject is estimated using Equation 6.

CBV(L)=weight(kg)*AVG_BV(L/kg) (6)

Where weight is the subject weight measured in kilograms and AVG_BV is the estimated average blood volume in L/kg which is determined for each subject using a modified version of Gilcher's Rule of Five (Gilcher 1996) and the body mass index (BMI) chart classification of weight categories The Body Mass Index (BMI) parameter is used as an assessment of body fatness and to place patients into weight categories. The BMI is calculated—measured in kilograms per meters squared—using Equation 7:

$$BMI ( kg \text{/} {m}^{2} ) = \frac{weight ( kg )}{{( height ( m ) )}^{2}} \qquad ( 7 )$$

Weight is the subject weight measured in kg and height is the subject height measured in meters.

Gilcher's Rule of Five is used as the primary method of estimating the AVG_BV in Equation 6 classifies male, female, and infant subjects into four categories (Obese, Thin, Normal, and Muscular) and determines an average blood volume for those subjects. In the modified version used in our model, infants are excluded and we do not account for subject muscularity. Subject calculated BMI is used to categorize subjects in a way that parallels the Gilcher's Rule of Five as shown in Table 2.

TABLE 2
Comparison of the BMI Chart and a modified version
Gilcher's Rule of Five utilized in the development
of the models described in other embodiments.

| | | Modified Gilcher's Rule of Five | | |
|---|---|---|---|---|
| BMI Index Chart | | Blood Volume (mL/kg of Body Weight) | | |
| BMI | Category | Classification | Male | Female |
| <18.5 | Underweight | Thin | 65 | 60 |
| 18.5-24.9 | Normal | Normal | 70 | 65 |
| ≧25 | Overweight-Obese | Obese | 60 | 55 |

In an embodiment, the normalized drug level obtained from Equation 1, Equation 2, and Equation 3 can be used in subsequent steps of the method, if any.

In an embodiment, Equation 1 is the most robust and preferred model used to determine whether the patients fall within the population of patients normally distributed around the standardized population mean.

In an embodiment, the distribution of transformed drug concentration data normalized using calculated blood volume and using Equation 1, or Equation 2, or Equation 3 resembles a Gaussian distribution (a normally distributed symmetric bell curved function). In this population distribution, the distribution is standardized with the mean of the resulting population therefore being set to zero. The fitted population distribution therefore has 68% of the data within +/−1 standard deviation, 95% of the data within +/−2 standard deviations and the other 5% greater than +/−2 standard deviations. In order to access patient compliance we say that 95% of the time, compliant patients can be expected to fall within 95% of the data hence within +/−2 standard deviations of the population mean. Based on the design of these models any patient within +/−2 standard deviations of the population mean is likely to be complaint with their drug dosage regimen and the closer they are to the population mean, the more closely they resemble the patients whose parameters (raw drug concentration measured in oral fluid , subject height, subject weight, subject gender, subject body mass index, subject lean body weight, subject body surface area, subject prescribed drug dosage, and subject calculated blood volume) resemble the mean of the population used to design the model. However, “compliant” is not a quantitative term in this respect and any patient that demonstrates data from oral fluid analysis which when mathematically transformed and normalized using calculated blood volume and using Equation 1, or Equation 2, or Equation 3 falls within +/−2 standard deviations of the mathematically transformed and normalized standard distribution is likely “compliant”.

Subjects with mathematically transformed and normalized drug concentrations which fall outside +/−2 standard deviations of the corresponding mathematically transformed and normalized drug distribution may or may not be “compliant” in their adherence to their prescribed drug regimen. For example, for those subjects falling outside of −2 standard deviations from the mean of the standard distribution, it may be that they are ultra rapid metabolizers and have cleared the drug from their blood volume (e.g., a CYP2D6 genetic issue), that they are not adherent; e.g. they are taking their drug less frequently than prescribed for any number of reasons such as expense, improved efficacy (less dose required), or in the worst case, they may be diverting their drug to a different use (e.g., for someone else, or for resale). On the other side, if their transformed and normalized drug concentration falls beyond +2 standard deviations from the mean of the standard distribution, it is possible that they are compliant but have very low metabolic rates (e.g., a different type of CYP2D6 genetic issue) leading to a buildup of drug in their blood. Other reasons for high transformed and normalized drug concentrations could well result from noncompliance including taking larger amounts of drug than prescribed. In any event, the results of the comparison to the standard distribution will assist the health care provider with identifying adherence issues and resolving those issue to the benefit of the patient.

In a related embodiment, one or a plurality of subjects are assigned to a population. As used herein a “plurality of subjects” refers to two or more subjects, for example about 2 subjects, about 3 subjects, about 4 subjects, about 5 subjects, about 6 subjects, about 7 subjects, about 8 subjects, about 9 subjects, about 10 subjects, about 15 subjects, about 20 subjects, about 25 subjects, about 30 subjects, about 35 subjects, about 40 subjects, about 45 subjects, about 50 subjects, about 55 subjects, about 60 subjects, about 65 subjects, about 70 subjects, about 75 subjects, about 80 subjects, about 85 subjects, about 90 subjects, about 95 subjects, about 100 subjects, about 110 subjects, about 120 subjects, about 130 subjects, about 140 subjects, about 150 subjects, about 160 subjects, about 170 subjects, about 180 subjects, about 190 subjects, about 200 subjects, about 225 subjects, about 250 subjects, about 275 subjects, about 300 subjects, about 325 subjects, about 350 subjects, about 375 subjects, about 400 subjects, about 425 subjects, about 450 subjects, about 475 subjects, about 500 subjects, about 525 subjects, about 550 subjects, about 575 subjects, about 600 subjects, about 625 subjects, about 650 subjects, about 675 subjects, about 700 subjects, about 725 subjects, about 750 subjects, about 775 subjects, about 800 subjects, about 825 subjects, about 850 subjects, about 875 subjects, about 900 subjects, about 925 subjects, about 950 subjects, about 975 subjects, about 1000 subjects, about 1250 subjects, about 1500 subjects, about 1750 subjects, about 2000 subjects, about 2250 subjects, about 2500 subjects, about 2750 subjects, about 3000 subjects, about 3500 subjects, about 4000 subjects, about 4500 subjects, about 5000 subjects, about 5500 subjects, about 6000 subjects, about 6500 subjects, about 7000 subjects, about 7500 subjects, about 8000 subjects, about 8500 subjects, about 9000 subjects, about 9500 subjects, or about 10000 subjects. As used herein with respect to a population, the term “subject” is synonymous with the term “member” and refers to an individual that has been assigned to the population. In one embodiment, subpopulations may be established for a plurality of daily doses of a drug.

In an embodiment, a plurality of subjects of a population are each prescribed the same daily dose of a drug. In another embodiment, a plurality of subjects assigned to one subpopulation are each prescribed a first daily dose of a drug while a plurality of subjects assigned to a second, different subpopulation are each prescribed a second, different daily dose of a drug. In an embodiment, a plurality of subjects assigned to a population or subpopulation are each prescribed a daily dose of a drug for a time sufficient to achieve steady state. The term “time sufficient to achieve steady state” refers to the amount of time required, given the pharmacokinetics of the particular drug and the dose administered to the subject, to establish a substantially constant concentration or level of the drug assuming the dose and the frequency of administrations remain substantially constant. The time sufficient to achieve steady state may be determined from literature or other information corresponding to the drug. For example, labels or package inserts for FDA approved drugs often include information regarding typical times sufficient to achieve steady state plasma concentrations from initial dosing. Other non-limiting means to determine the time sufficient to achieve steady state include experiment, laboratory studies, analogy to similar drugs with similar absorption and excretion characteristics, etc.

Assignment of subjects to a population or subpopulation may be accomplished by any method known to those skilled in the art. For example, subjects may be assigned randomly to one of a plurality of subpopulations. In an embodiment, subjects are screened for one or more parameters before or after being assigned to a population. For example, subjects featuring one or more parameters that may tend to affect fluid levels of a drug may be excluded from a population, may not be assigned to a population, may be assigned to one of a plurality of subpopulations, or may be removed from a population or subpopulation during or after a data collection phase of a study. Subjects may be excluded from a population based on the presence or absence of one or more exclusion criteria such as high opioid metabolism, low opioid metabolism, lab abnormalities, impaired kidney or liver function, use of drugs with overlapping metabolites on the same day, excessive body weight or minimal body weight, or an inconsistent schedule of medication administration, as non-limiting examples.

The method may be used in combination with any other method known to those skilled in the art for detecting a subject's potential non-compliance with a prescribed treatment protocol based on the normalized variations of the population used to create these models. Non-limiting examples of such methods include: interviews with the subject, oral fluid testing for the presence or absence of detectable levels of a drug, observation of the subject's behavior, appreciating reports of diversion of the subject's prescribed drug to others, etc.

In an embodiment, a method according to the present invention is used to reduce risk of drug misuse in a subject. In another embodiment, a method according to the present invention is used to confirm a subject's non-adherence to a chronic opioid therapy (COT) regimen. In yet another embodiment, a method according to the present invention provides a probability that a subject is non-compliant with a prescribed drug regimen. In an embodiment, a data point from the oral fluid testing of a subject is mathematically transformed and normalized to compare to a similarly transformed and normalized standard distribution to assess compliance with their prescribed dose. In another embodiment, the mathematically transformed and normalized standard distribution is obtained from a body of collected oral fluid test results.

In some embodiments, the present disclosure provides a method of determining a risk a subject is non-compliant with a prescribed drug regimen, the method comprising determining a prescribed daily dose of the drug, an age, a weight, a height and a gender associated with the subject; determining a concentration of a primary metabolite of the drug in an oral fluid sample of the subject; determining a transformed and normalized metabolite concentration as a function of at least the concentration of the primary metabolite, the age, the weight, the height and the gender of the subject; comparing the transformed and normalized metabolite concentration to transformed and normalized metabolite concentrations from a control population to provide a metabolite concentration variance; and determining the risk the subject is non-compliant as a function of at least the metabolite concentration variance. In some embodiments, the method further comprises determining a calculated blood volume associated with the subject, wherein the normalized metabolite concentration is determined as a function of at least the calculated blood volume. In some embodiments, the normalized metabolite concentration is determined as a function of at least the prescribed daily dose of the drug. In some embodiments, the normalized metabolite concentration is determined as a function of at least an adjustment factor associated with the drug. In some embodiments, the normalized metabolite concentration is determined as a function of a lean body weight associated with the subject. In some embodiments, the normalized metabolite concentration is determined as a function of a body surface area associated with the subject. In some embodiments, the normalized metabolite concentration is determined as a function of a logarithmic transformation of at least some combination of the prescribed daily dose of the drug, the age, the weight, the height and the gender associated with the subject. In some embodiments, the normalized metabolite concentration is determined as a function of a calculated blood volume, an adjustment factor associated with the drug, and a logarithmic transformation of the concentration of the primary metabolite of the drug in the oral fluid, a lean body weight, a body surface area associated with the subject, and the prescribed daily dose of the drug. In some embodiments, the normalized metabolite concentration is determined as a function of a calculated blood volume, an adjustment factor associated with the drug, and a logarithmic transformation of the concentration of the primary metabolite of the drug in the oral fluid, a lean body weight associated with the subject, and the prescribed daily dose of the drug. In some embodiments, the normalized metabolite concentration is determined as a function of a calculated blood volume, an adjustment factor associated with the drug, and a logarithmic transformation of the concentration of the primary metabolite of the drug in the oral fluid, a lean body weight, and a body surface area associated with the subject. In some embodiments, the logarithmic transformation is a natural logarithmic transformation. In some embodiments, the normalized metabolite concentration is determined according to Equation 1. In some embodiments, the normalized metabolite concentration is determined according to Equation 2. In some embodiments, the normalized metabolite concentration is determined according to Equation 3. In some embodiments, the normalized metabolite concentrations from a control population represent a Gaussian distribution. In some embodiments, the Gaussian distribution includes about 95% of the subject population within +/−2 standard deviations. In some embodiments, the Gaussian distribution includes about 68% of the subject population within +/−1 standard deviation. In some embodiments, the drug is selected from the group consisting of controlled-release oxycodone, oxycodone, controlled release morphine, morphine, extended release morphine, hydrocodone, methadone, and a combination of controlled-release oxycodone and oxycodone. In some embodiments, the primary metabolite comprises the drug. In some embodiments, the drug comprises an opioid or an antipsychotic drug. In some embodiments, the drug comprises a benzodiazepine and/or a benzodiazepine metabolite. In some embodiments, the drug comprises buprenorphine. In some embodiments, the drug comprises marijuana In some embodiments, the drug comprises an antidepressant. In some embodiments, the drug comprises an anticonvulsant. In some embodiments, the drug comprises an amphetamine derivative. In some embodiments, the drug comprises an attention deficit hyperactivity disorder (ADHD) drug. In some embodiments, the drug comprises an Autism spectrum disorder (ASD) drug. In some embodiments, the drug comprises methylphenidate. In some embodiments, the drug comprises dexamphetamine or lisdexamphetamine. In some embodiments, the drug comprises amphetamine or an isomer thereof.

In some embodiments, the present disclosure provides a method of generating a compliance report associated with a subject, the method comprising determining a prescribed daily dose of a drug associated with the subject; determining an age, a weight, and a gender associated with the subject; estimating a blood volume associated with the subject; obtaining an oral fluid sample associated with the subject; determining a concentration of a primary metabolite of the drug in the oral fluid of the subject; submitting the primary metabolite concentration to a rules engine to produce a rules engine output that describes a relationship between the primary metabolite concentration and the prescribed daily dose of the drug; and generating a compliance report comprising the rules engine output. In some embodiments, the relationship between the primary metabolite concentration and the prescribed daily dose of the drug comprises a statement indicating that the subject is compliant or non-compliant with the prescribed daily dose of the drug. In some embodiments, the rules engine output comprises a normalized metabolite concentration. In some embodiments, the rules engine includes a rule for normalizing the primary metabolite concentration as a function of at least the estimated blood volume associated with the subject. In some embodiments, the method further comprises determining a concentration of a secondary metabolite of the drug in the oral fluid of the subject; and submitting the secondary metabolite concentration to the rules engine to rules engine output.

In some embodiments, a method of the present disclosure includes correlating a primary metabolite concentration and/or a secondary metabolite concentration to normalized primary and/or secondary metabolite concentrations obtained from oral fluid associated with a subject population consisting of subjects who have been prescribed the same daily dose of the drug. In some embodiments, the transformed and normalized primary and/or secondary metabolite concentrations obtained from oral fluid associated with the subject population represent a Gaussian distribution. In some embodiments, the Gaussian distribution includes about 95% of the subject population within +/−2 standard deviations. In some embodiments, the Gaussian distribution includes about 68% of the subject population within +/−1 standard deviation.

In some embodiments, the normalized metabolite concentration is determined as a function of a logarithmic transformation of at least some combination of the prescribed daily dose of the drug, the age, the weight, the height and the gender associated with the subject. In some embodiments, the normalized metabolite concentration is determined as a function of a calculated blood volume, an adjustment factor associated with the drug, and a logarithmic transformation of the concentration of the primary metabolite of the drug in the oral fluid, a lean body weight, a body surface area associated with the subject, and the prescribed daily dose of the drug. In some embodiments, the normalized metabolite concentration is determined as a function of a calculated blood volume, an adjustment factor associated with the drug, and a logarithmic transformation of the concentration of the primary metabolite of the drug in the oral fluid, a lean body weight associated with the subject, and the prescribed daily dose of the drug. In some embodiments, the normalized metabolite concentration is determined as a function of a calculated blood volume, an adjustment factor associated with the drug, and a logarithmic transformation of the concentration of the primary metabolite of the drug in the oral fluid, a lean body weight, and a body surface area associated with the subject. In some embodiments, the logarithmic transformation is a natural logarithmic transformation. In some embodiments, the normalized metabolite concentration is determined according to Equation 1. In some embodiments, the normalized metabolite concentration is determined according to Equation 2. In some embodiments, the normalized metabolite concentration is determined according to Equation 3. In some embodiments, the normalized metabolite concentrations from a control population represent a Gaussian distribution. In some embodiments, the Gaussian distribution includes about 95% of the subject population within +/−2 standard deviations. In some embodiments, the Gaussian distribution includes about 68% of the subject population within +/−1 standard deviation. In some embodiments, the drug is selected from the group consisting of controlled-release oxycodone, oxycodone, controlled release morphine, morphine, extended release morphine, hydrocodone, methadone, and a combination of controlled-release oxycodone and oxycodone. In some embodiments, the primary metabolite is the drug. In some embodiments, the drug comprises an opioid or an antipsychotic drug. In some embodiments, the drug comprises a benzodiazepine and/or a benzodiazepine metabolite. In some embodiments, the drug comprises buprenorphine. In some embodiments, the drug comprises marijuana. In some embodiments, the drug comprises an antidepressant. In some embodiments, the drug comprises an anticonvulsant. In some embodiments, the drug comprises an amphetamine derivative. In some embodiments, the drug comprises an attention deficit hyperactivity disorder (ADHD) drug. In some embodiments, the drug comprises an Autism spectrum disorder (ASD) drug. In some embodiments, the drug comprises methylphenidate. In some embodiments, the drug comprises dexamphetamine or lisdexamphetamine. In some embodiments, the drug comprises amphetamine or an isomer thereof.

In some embodiments, the present disclosure provides a system for generating a compliance report associated with a subject, the system comprising an input device to receive a drug metabolite concentration, a prescribed daily dose of a drug, an age, a weight, and a gender associated with the subject; a memory for storing a normalization rule and the prescribed daily dose of the drug, the age, the weight, and the gender associated with the subject; a processor to estimate a blood volume associated with the subject, normalize the drug metabolite concentration based on the normalization rule, and generate a compliance report that describes a relationship between the drug metabolite concentration and the prescribed daily dose of the drug; and an output device to display the compliance report. In some embodiments, the relationship between the drug metabolite concentration and the prescribed daily dose of the drug comprises a statement indicating that the subject is compliant or non-compliant with the prescribed daily dose of the drug. In some embodiments, the normalization rule includes a rule for normalizing the drug metabolite concentration as a function of at least an estimated blood volume associated with the subject. In some embodiments, the input device receives a concentration of a secondary metabolite of the drug; the memory stores the concentration of the secondary metabolite of the drug; and the processor normalizes the secondary metabolite concentration based on the normalization rule. In some embodiments, the normalization rule comprises correlating the drug metabolite concentration and/or the secondary metabolite concentration to normalized drug and/or secondary metabolite concentrations obtained from oral fluid associated with a subject population consisting of subjects who have been prescribed the same daily dose of the drug. In some embodiments, the normalized drug and/or secondary metabolite concentrations obtained from oral fluid associated with the subject population represent a Gaussian distribution. In some embodiments, the Gaussian distribution includes about 95% of the subject population within +/−2 standard deviations. In some embodiments, the Gaussian distribution includes about 68% of the subject population within +/−1 standard deviation. In some embodiments, the normalization rule comprises obtaining a logarithmic transformation of at least some combination of the prescribed daily dose of the drug, the age, the weight, the height and the gender associated with the subject. In some embodiments, the normalization rule comprises normalizing the drug metabolite concentration as a function of a calculated blood volume, an adjustment factor associated with the drug, and a logarithmic transformation of the concentration of the primary drug metabolite concentration, a lean body weight, a body surface area associated with the subject, and the prescribed daily dose of the drug. In some embodiments, the normalization rule comprises normalizing the drug metabolite concentration as a function of a calculated blood volume, an adjustment factor associated with the drug, and a logarithmic transformation of the concentration of the primary metabolite of the drug in the oral fluid, a lean body weight associated with the subject, and the prescribed daily dose of the drug. In some embodiments, the normalization rule comprises normalizing the drug metabolite concentration as a function of a calculated blood volume, an adjustment factor associated with the drug, and a logarithmic transformation of the concentration of the primary metabolite of the drug in the oral fluid, a lean body weight, and a body surface area associated with the subject. In some embodiments, the logarithmic transformation is a natural logarithmic transformation. In some embodiments, the normalization rule comprises normalizing the drug metabolite concentration according to Equation 1. In some embodiments, the normalization rule comprises normalizing the drug metabolite concentration according to Equation 2. In some embodiments, the normalization rule comprises normalizing the drug metabolite concentration according to Equation 3. In some embodiments, the normalized drug metabolite concentrations from a control population represent a Gaussian distribution. In some embodiments, the Gaussian distribution includes about 95% of the subject population within +/−2 standard deviations. In some embodiments, the Gaussian distribution includes about 68% of the subject population within +/−1 standard deviation. In some embodiments, the drug is selected from the group consisting of controlled-release oxycodone, oxycodone, controlled release morphine, morphine, extended release morphine, hydrocodone, methadone, and a combination of controlled-release oxycodone and oxycodone. In some embodiments, the drug metabolite is the drug. In some embodiments, the drug comprises an opioid or an antipsychotic drug. In some embodiments, the drug comprises a benzodiazepine and/or a benzodiazepine metabolite. In some embodiments, the drug comprises buprenorphine. In some embodiments, the drug comprises marijuana. In some embodiments, the drug comprises an antidepressant. In some embodiments, the drug comprises an anticonvulsant. In some embodiments, the drug comprises an amphetamine derivative. In some embodiments, the drug comprises an attention deficit hyperactivity disorder (ADHD) drug. In some embodiments, the drug comprises an Autism spectrum disorder (ASD) drug. In some embodiments, the drug comprises methylphenidate. In some embodiments, the drug comprises dexamphetamine or lisdexamphetamine. In some embodiments, the drug comprises amphetamine or an isomer thereof. In some embodiments, the drug metabolite concentration and/or the secondary metabolite concentration are obtained from oral fluid associated with the subject.

In some embodiments, the present disclosure provides a computer readable medium storing instructions structured to cause a computing device to receive a drug metabolite concentration, a prescribed daily dose of a drug, an age, a weight, and a gender associated with the subject; store a normalization rule and the prescribed daily dose of the drug, the age, the weight, and the gender associated with the subject; estimate a blood volume associated with the subject; normalize the drug metabolite concentration based on the normalization rule; generate a compliance report that describes a relationship between the drug metabolite concentration and the prescribed daily dose of the drug; and display the compliance report. In some embodiments, the relationship between the drug metabolite concentration and the prescribed daily dose of the drug comprises a statement indicating that the subject is compliant or non-compliant with the prescribed daily dose of the drug. In some embodiments, the normalization rule includes a rule for normalizing the drug metabolite concentration as a function of at least the estimated blood volume associated with the subject. In some embodiments, the instructions further cause the computing device to receive a concentration of a secondary metabolite of the drug associated with subject and normalize the secondary metabolite concentration based on the normalization rule. In some embodiments, the normalization rule comprises correlating the drug metabolite concentration and/or the secondary metabolite concentration to normalized drug and/or secondary metabolite concentrations obtained from oral fluid associated with a subject population consisting of subjects who have been prescribed the same daily dose of the drug. In some embodiments, the normalized drug and/or secondary metabolite concentrations obtained from oral fluid associated with the subject population represent a Gaussian distribution. In some embodiments, the Gaussian distribution includes about 95% of the subject population within +/−2 standard deviations. In some embodiments, the Gaussian distribution includes about 68% of the subject population within +/−1 standard deviation. In some embodiments, the normalization rule comprises normalizing the drug concentration as a function of a logarithmic transformation of at least some combination of the prescribed daily dose of the drug, the age, the weight, the height and the gender associated with the subject. In some embodiments, the normalization rule comprises normalizing the drug metabolite concentration as a function of a calculated blood volume, an adjustment factor associated with the drug, and a logarithmic transformation of the concentration of the primary metabolite of the drug in the oral fluid, a lean body weight, a body surface area associated with the subject, and the prescribed daily dose of the drug. In some embodiments, the normalization rule comprises normalizing the drug metabolite concentration as a function of a calculated blood volume, an adjustment factor associated with the drug, and a logarithmic transformation of the concentration of the primary metabolite of the drug in the oral fluid, a lean body weight associated with the subject, and the prescribed daily dose of the drug. In some embodiments, the normalization rule comprises normalizing the drug metabolite concentration as a function of a calculated blood volume, an adjustment factor associated with the drug, and a logarithmic transformation of the concentration of the primary metabolite of the drug in the oral fluid, a lean body weight, and a body surface area associated with the subject. In some embodiments, the logarithmic transformation is a natural logarithmic transformation. In some embodiments, the normalization rule comprises normalizing the drug metabolite concentration according to Equation 1. In some embodiments, the normalization rule comprises normalizing the drug metabolite concentration according to Equation 2. In some embodiments, the normalization rule comprises normalizing the drug metabolite concentration according to Equation 3. In some embodiments, the normalized drug metabolite concentrations from a control population represent a Gaussian distribution. In some embodiments, the Gaussian distribution includes about 95% of the subject population within +/−2 standard deviations. In some embodiments, the Gaussian distribution includes about 68% of the subject population within +/−1 standard deviation. In some embodiments, the drug is selected from the group consisting of controlled-release oxycodone, oxycodone, controlled release morphine, morphine, extended release morphine, hydrocodone, methadone, and a combination of controlled-release oxycodone and oxycodone. In some embodiments, the primary metabolite comprises the drug. In some embodiments, the drug comprises an opioid or an antipsychotic drug. In some embodiments, the drug comprises a benzodiazepine and/or a benzodiazepine metabolite. In some embodiments, the drug comprises buprenorphine. In some embodiments, the drug comprises marijuana. In some embodiments, the drug comprises an antidepressant. In some embodiments, the drug comprises an anticonvulsant. In some embodiments, the drug comprises an amphetamine derivative. In some embodiments, the drug comprises an attention deficit hyperactivity disorder (ADHD) drug. In some embodiments, the drug comprises an Autism spectrum disorder (ASD) drug. In some embodiments, the drug comprises methylphenidate. In some embodiments, the drug comprises dexamphetamine or lisdexamphetamine. In some embodiments, the drug comprises amphetamine or an isomer thereof.

In some embodiments, the present disclosure provides a method of treating a subject, the method comprising administering a drug to the subject; determining an age, a weight, a height and a gender associated with the subject; determining a concentration of a primary metabolite of the drug in an oral fluid sample of the subject; determining a normalized metabolite concentration as a function of at least the concentration of the primary metabolite, the age, the weight, the height and the gender of the subject; and comparing the normalized metabolite concentration to normalized metabolite concentrations from a control population to provide a metabolite concentration variance. In some embodiments, the drug is administered according to a prescribed drug regimen associated with the subject. In some embodiments, the method further comprises determining a calculated blood volume associated with the subject, wherein the normalized metabolite concentration is determined as a function of at least the calculated blood volume. In some embodiments, the method further comprises determining a risk that the subject is non-compliant with the prescribed drug regimen as a function of at least the metabolite concentration variance. In some embodiments, the method further comprises discontinuing administering the drug to the subject if the risk that the subject is non-compliant exceeds a threshold risk value. In some embodiments, the method further comprises continuing administering the drug to the subject if the risk that the subject is non-compliant does not exceed a threshold risk value. In some embodiments, the method further comprises determining a concentration of a secondary metabolite of the drug in the oral fluid of the subject; and comparing the secondary metabolite concentration to normalized secondary drug metabolite concentrations from the control population to provide a secondary metabolite concentration variance. In some embodiments, the normalized metabolite and/or secondary metabolite concentrations obtained from oral fluid associated with the subject population represent a Gaussian distribution. In some embodiments, the Gaussian distribution includes about 95% of the subject population within +/−2 standard deviations. In some embodiments, the Gaussian distribution includes about 68% of the subject population within +/−1 standard deviation. In some embodiments, the normalized metabolite concentration is determined as a function of a logarithmic transformation of at least some combination of the prescribed daily dose of the drug, the age, the weight, the height and the gender associated with the subject. In some embodiments, the normalized metabolite concentration is determined as a function of a calculated blood volume, an adjustment factor associated with the drug, and a logarithmic transformation of the concentration of the metabolite of the drug in the oral fluid, a lean body weight, a body surface area associated with the subject, and the prescribed daily dose of the drug. In some embodiments, the normalized metabolite concentration is determined as a function of a calculated blood volume, an adjustment factor associated with the drug, and a logarithmic transformation of the concentration of the metabolite of the drug in the oral fluid, a lean body weight associated with the subject, and the prescribed daily dose of the drug. In some embodiments, the normalized metabolite concentration is determined as a function of a calculated blood volume, an adjustment factor associated with the drug, and a logarithmic transformation of the concentration of the metabolite of the drug in the oral fluid, a lean body weight, and a body surface area associated with the subject. In some embodiments, the logarithmic transformation is a natural logarithmic transformation. In some embodiments, the normalized metabolite concentration is determined according to Equation 1. In some embodiments, the normalized metabolite concentration is determined according to Equation 2. In some embodiments, the normalized metabolite concentration is determined according to Equation 3. In some embodiments, the normalized metabolite concentrations from a control population represent a Gaussian distribution. In some embodiments, the Gaussian distribution includes about 95% of the subject population within +/−2 standard deviations. In some embodiments, the Gaussian distribution includes about 68% of the subject population within +/−1 standard deviation. In some embodiments, the drug is selected from the group consisting of controlled-release oxycodone, oxycodone, controlled release morphine, morphine, extended release morphine, hydrocodone, methadone, and a combination of controlled-release oxycodone and oxycodone. In some embodiments, the primary metabolite comprises the drug. In some embodiments, the drug comprises an opioid or an antipsychotic drug. In some embodiments, the drug comprises a benzodiazepine and/or a benzodiazepine metabolite. In some embodiments, the drug comprises buprenorphine. In some embodiments, the drug comprises marijuana. In some embodiments, the drug comprises an antidepressant. In some embodiments, the drug comprises an anticonvulsant. In some embodiments, the drug comprises an amphetamine derivative. In some embodiments, the drug comprises an attention deficit hyperactivity disorder (ADHD) drug. In some embodiments, the drug comprises an Autism spectrum disorder (ASD) drug. In some embodiments, the drug comprises methylphenidate. In some embodiments, the drug comprises dexamphetamine or lisdexamphetamine. In some embodiments, the drug comprises amphetamine or an isomer thereof.

In the above description, various methods have been described. It will be apparent to one of ordinary skill in the art that each of these methods may be implemented, in whole or in part, by software, hardware, and/or firmware. If implemented, in whole or in part, by software, the software may be stored on and executed by a tangible medium such as a CD-ROM, a floppy disk, a hard drive, a digital versatile disk (DVD), a read-only memory (ROM), etc.

### EXAMPLES

The following examples are for illustrative purposes only and are not to be construed as limiting the scope of the invention in any respect whatsoever.

### Example 1

### Hydrocodone

A female subject with an age of 54 years, 150 days (54.41 years), a weight of 180 lbs, and height of 65 inches is prescribed a 30 mg daily dose of hydrocodone.

Then oral fluid from the subject is tested. The concentration of the primary metabolite (also referred to as the parent drug, i.e., hydrocodone) is 34 ng/ml.

Therefore, the transformed and normalized drug concentration is determined as follows using Equation1:

$${NORM}_{D\_CONC} = \frac{ln ( \frac{P\_MET * LBW * BSA}{D\_DOSE} )}{CBV} + ADJ\_A$$

Where LBW, BSA, and CBV are calculated using Equation 4, Equation 5, and Equation 6 respectively.

The value of LBW can be determined as follows:

$$LBW ( kg ) = fact\_a * weight ( kg ) - fact\_b * {( \frac{weight ( kg )}{100 * height ( m )} )}^{2} \qquad ( 4 )$$

Where fact_a equals 1.1 for Men and 1.07 for Women and fact_b equals 128 for Men and 148 for women. Weight is the subject weight measured in kg and height is the subject height in m. Hence,

$$LBW ( kg ) = 1.07 * ( \frac{180}{2.2} ) kg - 148 * {( \frac{( \frac{180}{2.2} ) kg}{( 100 * \frac{65}{39.37} ) m} )}^{2} = 51.198 kg$$

The value of BSA can be determined as follows:

$$BSA ( {m}^{2} ) = \sqrt{( \frac{height ( cm ) * weight ( kg )}{3600} )} \qquad ( 5 )$$

Weight is the subject weight measured in kg and height is the subject height measured in cm. Therefore,

$$BSA ( {m}^{2} ) = \sqrt{( \frac{( 65 * 2.54 ) cm * ( \frac{180}{2.2} ) kg}{3600} )} = 1.933 {m}^{2}$$

The value of patient BMI is determined to be 29.95 using Equation 7:

$$BMI ( kg \text{/} {m}^{2} ) = \frac{weight ( kg )}{{( height ( m ) )}^{2}} \qquad ( 7 )$$

which puts the patient into the Overweight to Obese category according to the BMI chart in Table 2. Furthermore, using the modified version of Gilcher's rule of five detailed in Table 2, this patient would be categorized as an obese female and hence would have an estimated average blood volume of 55 mL/kg.

The value of the CBV can be determined as follows:

CBV(L)=weight(kg)*AVG_BV(L/kg) (6)

Weight is the subject weight measured in kg and AVG_BV is the estimated average blood volume measured in L/kg.

$$CBV ( L ) = ( \frac{180}{2.2} ) kg * \frac{( \frac{55}{1000} ) L}{kg} = 5.318 L$$

This leads to

$${NORM}_{D\_CONC} = \frac{ln ( \frac{( 34 \times {10}^{- 9} ) kg \text{/} L * 51.198 kg * 1.933 {m}^{2}}{( 30 \times {10}^{- 6} ) kg} )}{5.318 L} + 0.154 = - 0.2514$$

This patient falls just outside the −1 standard deviation of our model described using Equation 1. Thus, this model would predict that this patient is compliant within +/−2 standard deviations compared to a transformed and normalized standard distribution and even more correctly, just outside +/−1 standard deviation compared to a transformed and normalized standard distribution.

### Example 2

### Hydrocodone

A male subject with an age of 35 years, 18 days (35.05 years), a weight of 225 lbs, and height of 69 inches is prescribed a 40 mg daily dose of hydrocodone.

Then oral fluid from the subject is tested. The concentration of the primary metabolite (also referred to as the parent drug, i.e., hydrocodone) in the oral fluid is 101 ng/m I.

Therefore, the normalized drug concentration is determined as follows using Equation 2:

$${NORM}_{D\_CONC} = \frac{ln ( \frac{P\_MET * LBW}{D\_DOSE} )}{CBV} + ADJ\_B$$

Where LBW and CBV are calculated using Equation 4 and Equation 6 respectively.

The value of LBW can be determined as follows:

$$LBW ( kg ) = fact\_a * weight ( kg ) - fact\_b * {( \frac{weight ( kg )}{100 * height ( m )} )}^{2} \qquad ( 4 )$$

Where fact_a equals 1.1 for Men and 1.07 for Women and fact_b equals 128 for Men and 148 for women. Weight is the subject weight measured in kg and height is the subject height in m. Hence,

$$LBW ( kg ) = 1.1 * ( \frac{225}{2.2} ) kg - 128 * {( \frac{( \frac{225}{2.2} ) kg}{( 100 * \frac{69}{39.37} ) m} )}^{2} = 68.912 kg$$

The value of patient BMI is determined to be 33.22 using Equation 7:

$$BMI ( kg \text{/} {m}^{2} ) = \frac{weight ( kg )}{{( height ( m ) )}^{2}} \qquad ( 7 )$$

which puts the patient into the Overweight to Obese category according to the BMI chart in Table 2. Furthermore, using the modified version of Gilcher's rule of five detailed in Table 2, this patient would be categorized as an obese male and hence would have an estimated average blood volume of 60mL/kg.

The value of the CBV can be determined as follows:

CBV(L)=weight(kg)*AVG_BV(L/kg) (6)

Weight is the subject weight measured in kg and AVG_BV is the estimated average blood volume measured in L/kg.

$$CBV ( L ) = ( \frac{225}{2.2} ) kg * \frac{( \frac{60}{1000} ) L}{kg} = 6.136 L$$

This leads to

$${NORM}_{D\_CONC} = \frac{ln ( \frac{( 101 \times {10}^{- 9} ) kg \text{/} L * 68.912 kg}{( 40 \times {10}^{- 6} ) kg} )}{6.136 L} + 0.276 = - 0.0275$$

This patient falls just outside the 0 standard deviation of our model described using Equation 2. Thus, these data would indicate compliance of this patient with his prescribed drug dosing paradigm.

### Example 3

### Oxycodone

A male subject with an age of 37 years, 77 days (37.21years), a weight of 254 lbs, and height of 71 inches is prescribed a 90 mg daily dose of oxycodone.

Then oral fluid from the subject is tested. The concentration of the primary metabolite (also referred to as the parent drug, i.e., oxycodone) in the oral fluid is 429 ng/ml.

Therefore, the normalized drug concentration is determined as follows using Equation1:

$${NORM}_{D\_CONC} = \frac{ln ( \frac{P\_MET * LBW * BSA}{D\_DOSE} )}{CBV} + ADJ\_A$$

Where LBW, BSA, and CBV are calculated using Equation 4, Equation 5, and Equation 6 respectively.

The value of LBW can be determined as follows:

$$LBW ( kg ) = fact\_a * weight ( kg ) - fact\_b * {( \frac{weight ( kg )}{100 * height ( m )} )}^{2} \qquad ( 4 )$$

Where fact_a equals 1.1 for Men and 1.07 for Women and fact_b equals 128 for Men and 148 for women. Weight is the subject weight measured in kg and height is the subject height in m. Hence,

$$LBW ( kg ) = 1.1 * ( \frac{254}{2.2} ) kg - 128 * {( \frac{( \frac{254}{2.2} ) kg}{( 100 * \frac{71}{39.37} ) m} )}^{2} = 74.537 kg$$

The value of BSA can be determined as follows:

$$BSA ( {m}^{2} ) = \sqrt{( \frac{height ( cm ) * weight ( kg )}{3600} )} \qquad ( 5 )$$

Weight is the subject weight measured in kg and height is the subject height measured in cm. Therefore,

$$BSA ( {m}^{2} ) = \sqrt{( \frac{( 71 * 2.54 ) cm * ( \frac{254}{2.2} ) kg}{3600} )} = 2.400 {m}^{2}$$

The value of patient BMI is determined to be 35.42 using Equation 7:

$$BMI ( kg \text{/} {m}^{2} ) = \frac{weight ( kg )}{{( height ( m ) )}^{2}} \qquad ( 7 )$$

which puts the patient into the Overweight to Obese category according to the BMI chart in Table 2. Furthermore, using the modified version of Gilcher's rule of five detailed in Table 2, this patient would be categorized as an obese male and hence would have an estimated average blood volume of 60mL/kg.

The value of the CBV can be determined as follows:

CBV(L)=weight(kg)*AVG_BV(L/kg) (6)

Weight is the subject weight measured in kg and AVG_BV is the estimated average blood volume measured in L/kg.

$$CBV ( L ) = ( \frac{254}{2.2} ) kg * \frac{( \frac{602}{1000} ) L}{kg} = 6.927 L$$

This leads to

$${NORM}_{D\_CONC} = \frac{ln ( \frac{( 429 \times {10}^{- 9} ) kg \text{/} L * 74.537 kg * 2.400 {m}^{2}}{( 90 \times {10}^{- 6} ) kg} )}{6.927 L} + 0.152 = 0.129$$

This patient falls approximately halfway between 0 standard deviation and +1 standard deviation of our model described using Equation 1. Thus, this model would predict that this patient is compliant within +/−2 standard deviations compared to a transformed normalized standard distribution and even more correctly, just outside +/−0.5 standard deviation compared to a transformed normalized standard distribution.

### Example 4

### Oxycodone

A female subject with an age of 30 years, 204.4 days (30.56 years), a weight of 113 lbs, and height of 64 inches is prescribed a 60 mg daily dose of hydrocodone.

Then oral fluid from the subject is tested. The concentration of the primary metabolite (also referred to as the parent drug, i.e., oxycodone) in the oral fluid is 50 ng/ml.

Therefore, the normalized drug concentration is determined as follows using Equation 2:

$${NORM}_{D\_CONC} = \frac{ln ( \frac{P\_MET * LBW}{D\_DOSE} )}{CBV} + ADJ\_B$$

Where LBW and CBV are calculated using Equation 4 and Equation 6 respectively.

The value of LBW can be determined as follows:

$$LBW ( kg ) = fact\_a * weight ( kg ) - fact\_b * {( \frac{weight ( kg )}{100 * height ( m )} )}^{2} \qquad ( 4 )$$

Where fact_a equals 1.1 for Men and 1.07 for Women and fact_b equals 128 for Men and 148 for women. Weight is the subject weight measured in kg and height is the subject height in m. Hence,

$$LBW ( kg ) = 1.07 * ( \frac{117}{2.2} ) kg - 148 * {( \frac{( \frac{117}{2.2} ) kg}{( 100 * \frac{64}{39.37} ) m} )}^{2} = 40.183 kg$$

The value of patient BMI is determined to be 19.40 using Equation 7:

$$BMI ( kg \text{/} {m}^{2} ) = \frac{weight ( kg )}{{( height ( m ) )}^{2}} \qquad ( 7 )$$

which puts the patient into the Normal category according to the BMI chart in Table 2. Furthermore, using the modified version of Gilcher's rule of five detailed in Table 2, this patient would be categorized as a normal female and hence would have an estimated average blood volume of 65mL/kg.

The value of the CBV can be determined as follows:

CBV(L)=weight(kg)*AVG_BV(L/kg) (6)

Weight is the subject weight measured in kg and AVG_BV is the estimated average blood volume measured in L/kg.

$$CBV ( L ) = ( \frac{225}{2.2} ) kg * \frac{( \frac{65}{1000} ) L}{kg} = 3.339 L$$

This leads to

$${NORM}_{D\_CONC} = \frac{ln ( \frac{( 50 \times {10}^{- 9} ) kg \text{/} L * 40.183 kg}{( 60 \times {10}^{- 6} ) kg} )}{3.339 L} + 0.279 = - 0.9895$$

This patient falls just outside (i.e., below) the +/−2 (e.g., -2 std dev) standard deviation of our model described using Equation 2. Thus, these data would indicate possible non-compliance of this patient with her prescribed drug dosing paradigm. Such non-compliance could take the form of less frequent dosing than prescribed (i.e., every other day vs every day), pill splitting to extend prescription length, or diversion to other uses and/or people (Cole, 2001).

### Example 5

### Test of a Population of 50 Hydrocodone Patient Samples

The results (drug concentration of the primary metabolite), demographic information (gender, weight, height, and age), and the prescribed dosage of hydrocodone for fifty randomly selected patients—not included in the patient population used to design the models—were used to assess the validity and robustness of the models. The corresponding data is presented in Table 3.

TABLE 3
Oral fluid drug concentrations, demographic information
(gender, weight, height, and age), and the prescribed dosage
of hydrocodone for the sample patient population.

| Sample | | | | | Daily | Hydrocodone |
|---|---|---|---|---|---|---|
| Patient | | Weight | Height | Age | Dose | in Oral Fluid |
| # | Gender | (lbs) | (inches) | (yrs) | (mg) | (ng/mL) |
| 1 | F | 190 | 64 | 65.41 | 15 | 294 |
| 2 | M | 258 | 70 | 42.00 | 40 | 293 |
| 3 | F | 138 | 65 | 85.36 | 30 | 292 |
| 4 | F | 158 | 62 | 85.36 | 22.5 | 109 |
| 5 | M | 295 | 70 | 74.78 | 30 | 329 |
| 6 | F | 115 | 63 | 49.72 | 60 | 1755 |
| 7 | M | 189 | 76 | 52.23 | 30 | 1883 |
| 8 | F | 116 | 65 | 49.66 | 120 | 2178 |
| 9 | M | 182 | 72 | 36.90 | 30 | 684 |
| 10 | M | 189 | 67 | 32.74 | 40 | 3858 |
| 11 | F | 163 | 65 | 24.85 | 120 | 234 |
| 12 | F | 123 | 60 | 61.45 | 30 | 1764 |
| 13 | F | 192 | 61 | 36.74 | 30 | 164 |
| 14 | M | 146 | 75 | 42.52 | 40 | 167 |
| 15 | F | 143 | 65 | 43.50 | 40 | 582 |
| 16 | F | 230 | 64 | 64.16 | 80 | 32 |
| 17 | F | 204 | 68 | 47.66 | 30 | 481 |
| 18 | M | 169 | 68 | 62.85 | 80 | 685 |
| 19 | F | 254 | 63 | 40.46 | 22.5 | 1989 |
| 20 | M | 383 | 76 | 49.74 | 80 | 178 |
| 21 | F | 245 | 65 | 53.87 | 40 | 255 |
| 22 | F | 240 | 64 | 69.69 | 15 | 300 |
| 23 | F | 157 | 66 | 51.86 | 60 | 106 |
| 24 | M | 500 | 68 | 27.25 | 40 | 126 |
| 25 | F | 167 | 63 | 81.05 | 40 | 35 |
| 26 | M | 243 | 76 | 33.14 | 40 | 860 |
| 27 | M | 285 | 76 | 28.19 | 40 | 893 |
| 28 | F | 109 | 65 | 38.83 | 30 | 845 |
| 29 | F | 165 | 64 | 75.22 | 30 | 2335 |
| 30 | M | 275 | 64 | 51.10 | 60 | 657 |
| 31 | F | 234 | 64 | 30.58 | 5 | 36 |
| 32 | M | 223 | 66 | 53.95 | 30 | 2271 |
| 33 | F | 284 | 67 | 95.52 | 40 | 262 |
| 34 | M | 190 | 69 | 61.85 | 60 | 76 |
| 35 | F | 246 | 66 | 51.91 | 20 | 813 |
| 36 | M | 135 | 63 | 75.55 | 60 | 677 |
| 37 | F | 220 | 64 | 25.67 | 22.5 | 93 |
| 38 | M | 258 | 74 | 59.86 | 40 | 245 |
| 39 | M | 220 | 68 | 53.35 | 40 | 32 |
| 40 | F | 190 | 64 | 65.42 | 15 | 300 |
| 41 | M | 191 | 73 | 36.41 | 40 | 1102 |
| 42 | M | 241 | 74 | 64.01 | 40 | 140 |
| 43 | F | 214 | 62 | 36.96 | 10 | 90 |
| 44 | F | 227 | 65 | 64.79 | 40 | 397 |
| 45 | F | 161 | 59 | 57.47 | 30 | 879 |
| 46 | F | 242 | 68 | 50.08 | 60 | 115 |
| 47 | F | 242 | 63 | 53.48 | 30 | 198 |
| 48 | F | 122 | 60 | 94.56 | 20 | 233 |
| 49 | M | 150 | 74 | 57.73 | 30 | 2323 |
| 50 | M | 165 | 64 | 85.82 | 22.5 | 50 |

The normalized drug concentrations for all patients were calculated using Equation 1, or Equation 2, or Equation 3 following the calculation of LBW, BSA, BMI, AVG_BV and CBV according to Equations 4 through Equations 7 detailed in another embodiment. The calculated results for Equation 1, Equation 2, and Equation 3 are presented in Table 4. The raw normalized results are presented along with a description of whether the result was within +/−1 standard deviation, +/−2 standard deviations, out outside the range. For patient results within +/−1 standard deviation, these patients are very likely to be in compliance with their regimen. For patient results within +/−2 standard deviations, these patients are likely to be in compliance with their regimen. For patient results that fall outside the range—with the value of the normalized drug concentration greater than +/−2 standard deviations—are possibly non-compliant with their regimen or may have some condition not considered by the model hence causing them to not fall within at least the 95% range of the model (e.g., Rapid or absence of metabolic genetic machinery (CYP2D6))

TABLE 4
Normalized drug concentrations determined from Equation 1, Equation
2, or Equation 3 for hydrocodone as well as the range of the
result as a function of standard deviations from the mean.

| Sample | | | | | | |
|---|---|---|---|---|---|---|
| Patient | Equation | Equation | Equation | Equation 1 | Equation 2 | Equation 3 |
| # | 1 | 2 | 3 | Result | Result | Result |
| 1 | 0.29 | 0.27 | −0.08 | Within +/−2 Std | Within +/−2 Std | Within +/−1 Std |
| 2 | 0.18 | 0.19 | 0.71 | Within +/−1 Std | Within +/−1 Std | Within +/−2 Std |
| 3 | 0.08 | 0.08 | −0.51 | Within +/−1 Std | Within +/−1 Std | Within +/−1 Std |
| 4 | −0.05 | −0.05 | −0.38 | Within +/−1 Std | Within +/−1 Std | Within +/−1 Std |
| 5 | 0.24 | 0.25 | 0.91 | Within +/−2 Std | Within +/−2 Std | Within +/−2 Std |
| 6 | 0.32 | 0.32 | −0.58 | Within +/−2 Std | Within +/−2 Std | Within +/−1 Std |
| 7 | 0.52 | 0.52 | 0.75 | Outside the | Outside the | Within +/−2 Std |
| | | | | Range | Range | |
| 8 | 0.19 | 0.19 | −0.48 | Within +/−1 Std | Within +/−1 Std | Within +/−1 Std |
| 9 | 0.34 | 0.34 | 0.51 | Within +/−2 Std | Within +/−2 Std | Within +/−1 Std |
| 10 | 0.56 | 0.57 | 0.84 | Outside the | Outside the | Within +/−2 Std |
| | | | | Range | Range | |
| 11 | −0.21 | −0.21 | −0.12 | Within +/−2 Std | Within +/−1 Std | Within +/−1 Std |
| 12 | 0.50 | 0.51 | −0.40 | Outside the | Outside the | Within +/−1 Std |
| | | | | Range | Range | |
| 13 | 0.00 | −0.01 | −0.20 | Within +/−1 Std | Within +/−1 Std | Within +/−1 Std |
| 14 | −0.04 | −0.05 | −0.42 | Within +/−1 Std | Within +/−1 Std | Within +/−1 Std |
| 15 | 0.18 | 0.18 | −0.25 | Within +/−1 Std | Within +/−1 Std | Within +/−1 Std |
| 16 | −0.40 | −0.40 | −0.07 | Within +/−2 Std | Within +/−2 Std | Within +/−1 Std |
| 17 | 0.27 | 0.26 | 0.20 | Within +/−2 Std | Within +/−2 Std | Within +/−1 Std |
| 18 | 0.14 | 0.15 | 0.35 | Within +/−1 Std | Within +/−1 Std | Within +/−1 Std |
| 19 | 0.50 | 0.50 | 0.78 | Outside the | Outside the | Within +/−2 Std |
| | | | | Range | Range | |
| 20 | 0.10 | 0.12 | 1.16 | Within +/−1 Std | Within +/−1 Std | Within +/−2 Std |
| 21 | 0.10 | 0.10 | 0.41 | Within +/−1 Std | Within +/−1 Std | Within +/−1 Std |
| 22 | 0.28 | 0.28 | 0.39 | Within +/−2 Std | Within +/−2 Std | Within +/−1 Std |
| 23 | −0.25 | −0.25 | −0.38 | Within +/−2 Std | Within +/−2 Std | Within +/−1 Std |
| 24 | 0.06 | 0.10 | 1.28 | Within +/−1 Std | Within +/−1 Std | Outside the |
| | | | | | | Range |
| 25 | −0.37 | −0.37 | −0.46 | Within +/−2 Std | Within +/−2 Std | Within +/−1 Std |
| 26 | 0.33 | 0.35 | 0.99 | Within +/−2 Std | Within +/−2 Std | Within +/−2 Std |
| 27 | 0.35 | 0.36 | 1.02 | Within +/−2 Std | Within +/−2 Std | Within +/−2 Std |
| 28 | 0.32 | 0.31 | −1.22 | Within +/−2 Std | Within +/−2 Std | Outside the |
| | | | | | | Range |
| 29 | 0.55 | 0.55 | 0.37 | Outside the | Outside the | Within +/−1 Std |
| | | | | Range | Range | |
| 30 | 0.21 | 0.22 | 0.88 | Within +/−2 Std | Within +/−2 Std | Within +/−2 Std |
| 31 | 0.11 | 0.10 | −0.01 | Within +/−1 Std | Within +/−1 Std | Within +/−1 Std |
| 32 | 0.54 | 0.54 | 0.79 | Outside the | Outside the | Within +/−2 Std |
| | | | | Range | Range | |
| 33 | 0.13 | 0.13 | 0.66 | Within +/−1 Std | Within +/−1 Std | Within +/−2 Std |
| 34 | −0.15 | −0.14 | 0.21 | Within +/−1 Std | Within +/−1 Std | Within +/−1 Std |
| 35 | 0.41 | 0.40 | 0.61 | Within +/−2 Std | Within +/−2 Std | Within +/−2 Std |
| 36 | 0.12 | 0.14 | −0.17 | Within +/−1 Std | Within +/−1 Std | Within +/−1 Std |
| 37 | 0.00 | −0.01 | 0.02 | Within +/−1 Std | Within +/−1 Std | Within +/−1 Std |
| 38 | 0.17 | 0.17 | 0.70 | Within +/−1 Std | Within +/−1 Std | Within +/−2 Std |
| 39 | −0.21 | −0.21 | 0.07 | Within +/−1 Std | Within +/−2 Std | Within +/−1 Std |
| 40 | 0.29 | 0.28 | −0.08 | Within +/−2 Std | Within +/−2 Std | Within +/−1 Std |
| 41 | 0.37 | 0.38 | 0.67 | Within +/−2 Std | Within +/−2 Std | Within +/−2 Std |
| 42 | 0.08 | 0.08 | 0.51 | Within +/−1 Std | Within +/−1 Std | Within +/−1 Std |
| 43 | 0.12 | 0.12 | −0.06 | Within +/−1 Std | Within +/−1 Std | Within +/−1 Std |
| 44 | 0.17 | 0.16 | 0.35 | Within +/−1 Std | Within +/−1 Std | Within +/−1 Std |
| 45 | 0.34 | 0.33 | −0.28 | Within +/−2 Std | Within +/−2 Std | Within +/−1 Std |
| 46 | −0.08 | −0.09 | 0.28 | Within +/−1 Std | Within +/−1 Std | Within +/−1 Std |
| 47 | 0.09 | 0.09 | 0.33 | Within +/−1 Std | Within +/−1 Std | Within +/−1 Std |
| 48 | 0.05 | 0.06 | −0.98 | Within +/−1 Std | Within +/−1 Std | Within +/−2 Std |
| 49 | 0.60 | 0.59 | 0.38 | Outside the | Outside the | Within +/−1 Std |
| | | | | Range | Range | |
| 50 | −0.14 | −0.12 | −0.21 | Within +/−1 Std | Within +/−1 Std | Within +/−1 Std |

Using Equation 3, the data approximates the expected normal distribution pattern with approximately 66% falling within +/−1 standard deviation (˜66%), 94% falling with +/−2 standard deviations (˜96%) and 6% falling outside the +/−2 standard deviation range (˜4%). The model that corresponds to Equation 3, however, does not account for the dosage of the drug that the patient has been prescribed and is less discriminating than either Equation 1 or Equation 2.

In both Equation 1 and Equation 2, each of which accounts for the dosage of the drug that the patient been prescribed, 50% of the patients fall within +/−1 standard deviation, 86% fall within +/−2 standard deviations and 14% fall outside the +/−2 standard deviation range. If we examine the data presented in Table 3, it is evident that for this sample population, many of the patients determined to be outside the range (detailed in Table 4) have measured drug concentration that is significantly greater than other patients who were prescribed similar dosages. Hence while it is likely that these patients are non-complaint with their drug regimen, it is possible that some of these patients may have conditions not considered by the model, causing them to not fall within at least the 95% range of the model (e.g., absence of metabolic genetic machinery (CYP2D6)).

### Example 6

### Test of a Population of 50 Oxycodone Patient Samples

The results (drug concentration of the primary metabolite), demographic information (gender, weight, height, and age), and the prescribed dosage of oxycodone for fifty randomly selected patients—not included in the patient population used to design the models—were used to assess the validity and robustness of the models. The corresponding data is presented in Table 5.

TABLE 5
Oral fluid drug concentrations, demographic information
(gender, weight, height, and age), and the prescribed dosage
of oxycodone for the sample patient population.

| Sample | | | | | Daily | Oxycodone |
|---|---|---|---|---|---|---|
| Patient | | Weight | Height | Age | Dose | in Oral Fluid |
| # | Gender | (lbs) | (inches) | (yrs) | (mg) | (ng/mL) |
| 1 | F | 73 | 53 | 82.14 | 25 | 164 |
| 2 | F | 106 | 60 | 58.66 | 120 | 126 |
| 3 | F | 108 | 58 | 60.30 | 15 | 300 |
| 4 | F | 119 | 63 | 45.97 | 30 | 90 |
| 5 | F | 217 | 66 | 45.17 | 240 | 32 |
| 6 | F | 180 | 62 | 82.73 | 40 | 294 |
| 7 | M | 180 | 70 | 57.89 | 22.5 | 293 |
| 8 | M | 185 | 68 | 66.07 | 40 | 292 |
| 9 | M | 213 | 72 | 44.69 | 120 | 109 |
| 10 | F | 170 | 64 | 52.90 | 120 | 329 |
| 11 | M | 216 | 70 | 62.42 | 120 | 684 |
| 12 | M | 189 | 67 | 32.74 | 120 | 234 |
| 13 | M | 129 | 65 | 40.42 | 240 | 582 |
| 14 | M | 208 | 68 | 65.68 | 180 | 685 |
| 15 | M | 217 | 72 | 54.16 | 90 | 178 |
| 16 | F | 209 | 67 | 70.50 | 55 | 255 |
| 17 | M | 178 | 73 | 67.77 | 40 | 69 |
| 18 | M | 243 | 76 | 33.14 | 120 | 35 |
| 19 | F | 166 | 63 | 33.11 | 90 | 893 |
| 20 | F | 157 | 65 | 31.64 | 120 | 657 |
| 21 | M | 200 | 65 | 36.15 | 15 | 76 |
| 22 | M | 199 | 70 | 32.00 | 120 | 93 |
| 23 | M | 200 | 64 | 41.33 | 90 | 245 |
| 24 | M | 188 | 74 | 35.44 | 30 | 32 |
| 25 | M | 225 | 72 | 29.82 | 200 | 300 |
| 26 | M | 232 | 72 | 73.70 | 90 | 397 |
| 27 | M | 144 | 67 | 71.77 | 210 | 879 |
| 28 | F | 170 | 63 | 38.09 | 120 | 2335 |
| 29 | M | 204 | 70 | 48.88 | 10 | 140 |
| 30 | M | 197 | 72 | 46.97 | 30 | 481 |
| 31 | F | 234 | 68 | 40.33 | 15 | 860 |
| 32 | M | 208 | 68 | 29.34 | 80 | 36 |
| 33 | M | 141 | 68 | 44.48 | 120 | 2271 |
| 34 | F | 132 | 68 | 41.10 | 30 | 813 |
| 35 | F | 187 | 59 | 33.63 | 15 | 1102 |
| 36 | F | 208 | 70 | 59.73 | 420 | 4567 |
| 37 | F | 115 | 63 | 69.97 | 60 | 845 |
| 38 | M | 236 | 72 | 50.08 | 360 | 1256 |
| 39 | M | 285 | 76 | 28.19 | 120 | 262 |
| 40 | M | 265 | 74 | 56.10 | 260 | 677 |
| 41 | F | 120 | 65 | 57.03 | 40 | 198 |
| 42 | M | 195 | 74 | 39.27 | 60 | 1755 |
| 43 | M | 217 | 73 | 44.72 | 120 | 1883 |
| 44 | M | 189 | 76 | 52.23 | 120 | 2178 |
| 45 | M | 275 | 68 | 53.11 | 220 | 3858 |
| 46 | M | 192 | 76 | 35.42 | 120 | 1764 |
| 47 | F | 124 | 64 | 45.52 | 60 | 167 |
| 48 | M | 186 | 68 | 45.88 | 120 | 1989 |
| 49 | F | 170 | 60 | 60.22 | 120 | 106 |
| 50 | M | 242 | 71 | 44.30 | 90 | 1157 |

The normalized drug concentrations for all patients were calculated using Equation 1, Equation 2, and Equation 3 following the calculation of LBW, BSA, BMI, AVG_BV and CBV according to Equations 4 through Equations 7 detailed in another embodiment. The calculated results for Equation 1, Equation 2, and Equation 3 are presented in Table 4. The raw normalized results are presented along with a description of whether the result was within +/−1 standard deviation, +/−2 standard deviations, out outside the range. For patient results within +/−1 standard deviation, these patients are very likely to be in compliance with their regimen. For patient results within +/−2 standard deviations, these patients are likely to be in compliance with their regimen. For patient results that fall outside the range—with the value of the normalized drug concentration greater than +/−2 standard deviations—are likely to be in non-compliance with their regimen or may have some condition not considered by the model hence causing them to not fall within at least the 95% range of the model.

TABLE 6
Normalized drug concentrations determined from Equation 1, Equation
2, and Equation 3 for Oxocodone as well as the range of the result
as a function of standard deviations from the mean.

| Sample | | | | | | |
|---|---|---|---|---|---|---|
| Patient | Equation | Equation | Equation | Equation 1 | Equation 2 | Equation 3 |
| # | 1 | 2 | 3 | Result | Result | Result |
| 1 | −0.67 | −0.60 | −4.10 | Outside the | Outside the | Outside the |
| | | | | Range | Range | Range |
| 2 | −0.77 | −0.76 | −1.76 | Outside the | Outside the | Outside the |
| | | | | Range | Range | Range |
| 3 | 0.16 | 0.18 | −1.42 | Within +/−1 Std | Within +/−1 Std | Outside the |
| | | | | | | Range |
| 4 | −0.32 | −0.32 | −1.38 | Within +/−2 Std | Within +/−2 Std | Outside the |
| | | | | | | Range |
| 5 | −0.62 | −0.63 | −0.25 | Outside the | Outside the | Within +/−1 Std |
| | | | | Range | Range | |
| 6 | −0.09 | −0.23 | −0.29 | Within +/−1 Std | Within +/−1 Std | Within +/−1 Std |
| 7 | 0.09 | −0.03 | 0.27 | Within +/−1 Std | Within +/−1 Std | Within +/−1 Std |
| 8 | −0.02 | −0.13 | 0.31 | Within +/−1 Std | Within +/−1 Std | Within +/−1 Std |
| 9 | −0.14 | −0.13 | 0.43 | Within +/−1 Std | Within +/−1 Std | Within +/−1 Std |
| 10 | −0.12 | −0.12 | −0.02 | Within +/−1 Std | Within +/−1 Std | Within +/−1 Std |
| 11 | 0.13 | 0.12 | 0.49 | Within +/−1 Std | Within +/−1 Std | Within +/−1 Std |
| 12 | −0.08 | −0.07 | 0.31 | Within +/−1 Std | Within +/−1 Std | Within +/−1 Std |
| 13 | −0.25 | −0.24 | −0.38 | Within +/−1 Std | Within +/−1 Std | Within +/−1 Std |
| 14 | 0.04 | 0.03 | 0.42 | Within +/−1 Std | Within +/−1 Std | Within +/−1 Std |
| 15 | −0.02 | 0.00 | 0.53 | Within +/−1 Std | Within +/−1 Std | Within +/−1 Std |
| 16 | 0.04 | 0.02 | 0.06 | Within +/−1 Std | Within +/−1 Std | Within +/−1 Std |
| 17 | −0.11 | −0.11 | 0.00 | Within +/−1 Std | Within +/−1 Std | Within +/−1 Std |
| 18 | −0.22 | −0.21 | 0.51 | Within +/−1 Std | Within +/−1 Std | Within +/−1 Std |
| 19 | 0.12 | 0.13 | 0.12 | Within +/−1 Std | Within +/−1 Std | Within +/−1 Std |
| 20 | −0.01 | −0.01 | −0.05 | Within +/−1 Std | Within +/−1 Std | Within +/−1 Std |
| 21 | 0.07 | 0.06 | −0.07 | Within +/−1 Std | Within +/−1 Std | Within +/−1 Std |
| 22 | −0.20 | −0.19 | 0.27 | Within +/−1 Std | Within +/−1 Std | Within +/−1 Std |
| 23 | −0.05 | −0.05 | 0.14 | Within +/−1 Std | Within +/−1 Std | Within +/−1 Std |
| 24 | −0.16 | −0.16 | −0.01 | Within +/−1 Std | Within +/−1 Std | Within +/−1 Std |
| 25 | −0.08 | −0.08 | 0.44 | Within +/−1 Std | Within +/−1 Std | Within +/−1 Std |
| 26 | 0.11 | 0.10 | 0.53 | Within +/−1 Std | Within +/−1 Std | Within +/−1 Std |
| 27 | −0.05 | −0.05 | 0.00 | Within +/−1 Std | Within +/−1 Std | Within +/−1 Std |
| 28 | 0.28 | 0.26 | 0.06 | Within +/−2 Std | Within +/−1 Std | Within +/−1 Std |
| 29 | 0.26 | 0.27 | 0.38 | Within +/−1 Std | Within +/−2 Std | Within +/−1 Std |
| 30 | 0.29 | 0.29 | 0.52 | Within +/−2 Std | Within +/−2 Std | Within +/−1 Std |
| 31 | 0.50 | 0.48 | 0.50 | Within +/−2 Std | Within +/−2 Std | Within +/−1 Std |
| 32 | −0.34 | −0.34 | −0.10 | Within +/−2 Std | Within +/−2 Std | Within +/−1 Std |
| 33 | 0.28 | 0.28 | 0.16 | Within +/−2 Std | Within +/−2 Std | Within +/−1 Std |
| 34 | 0.35 | 0.34 | −0.43 | Within +/−2 Std | Within +/−2 Std | Within +/−1 Std |
| 35 | 0.53 | 0.53 | 0.06 | Within +/−2 Std | Within +/−2 Std | Within +/−1 Std |
| 36 | 0.05 | −0.07 | 0.84 | Within +/−1 Std | Within +/−1 Std | Within +/−2 Std |
| 37 | 0.11 | 0.11 | −0.85 | Within +/−1 Std | Within +/−1 Std | Within +/−2 Std |
| 38 | 0.07 | 0.07 | 0.74 | Within +/−1 Std | Within +/−1 Std | Within +/−2 Std |
| 39 | 0.06 | 0.06 | 0.80 | Within +/−1 Std | Within +/−1 Std | Within +/−2 Std |
| 40 | 0.06 | 0.06 | 0.82 | Within +/−1 Std | Within +/−1 Std | Within +/−2 Std |
| 41 | −0.16 | −0.16 | −1.12 | Within +/−1 Std | Within +/−1 Std | Within +/−2 Std |
| 42 | 0.39 | 0.39 | 0.72 | Within +/−2 Std | Within +/−2 Std | Within +/−2 Std |
| 43 | 0.29 | 0.30 | 0.88 | Within +/−2 Std | Within +/−2 Std | Within +/−2 Std |
| 44 | 0.32 | 0.32 | 0.71 | Within +/−2 Std | Within +/−2 Std | Within +/−2 Std |
| 45 | 0.30 | 0.31 | 1.08 | Within +/−2 Std | Within +/−2 Std | Within +/−2 Std |
| 46 | 0.28 | 0.28 | 0.70 | Within +/−2 Std | Within +/−2 Std | Within +/−2 Std |
| 47 | −0.30 | −0.30 | −1.06 | Within +/−2 Std | Within +/−2 Std | Within +/−2 Std |
| 48 | 0.28 | 0.28 | 0.65 | Within +/−2 Std | Within +/−2 Std | Within +/−2 Std |
| 49 | −0.47 | −0.48 | −0.70 | Within +/−2 Std | Within +/−2 Std | Within +/−2 Std |
| 50 | 0.27 | 0.27 | 0.76 | Within +/−2 Std | Within +/−2 Std | Within +/−2 Std |

Using Equation 3, the data closely mirrors the expected normal distribution pattern with approximately 62% falling within +/−1 standard deviation, 92% falling within +/−2 standard deviations and 8% falling outside the +/−2 standard deviation range. The model that corresponds to Equation 3, however, does not account for the dosage of the drug that the patient has been prescribed.

In both Equation 1 and Equation 2, each of which accounts for the dosage of the drug that the patient been prescribed, 60% of the patients fall within +/−1 standard deviation, 94% fall with +/−2 standard deviations and 6% fall outside the +/−2 standard deviation range. The 6% of patients who fall outside the +/−2 standard deviation range are very likely non-complaint with their drug regimen or may have some condition not considered by the model hence causing them to not fall within at least the 95% range of the model.

### REFERENCES

1. A. Collins, J. Bourland and R. Backer. Disposition of oxycodone in oral fluid. Poster Presentation at Society of Forensic Toxicologists Annual Meeting, 2009.
2. E. J. Cone and M. Heustis. Interpretation of Oral Fluid Tests for Drugs of Abuse, Ann N Y Acad Sci. 1098: 51-103. (2007).
3. T. Conermann, A. Gozalia, A. J. Kabazie, C. Moore, K. Miller, M. Fetsch and D. Irvan. Utility of oral fluid in compliance monitoring of opioid medication. Pain Physician. 17: 63-70. (2014).
4. V. Vindenes, B. Yttredal, E. L. Oiestad, H. Waal, J. P. Bernard, J. G. Morland and A. S. Christophersen. Oral fluid is a viable alternative for monitoring drug abuse: detection of drugs in oral fluid by liquid chromatography-tandem mass spectrometry and comparison to the results from urine samples from patients treated with methadone or buprenorphine. J. Anal. Toxicol. 35: 32-39. (2011).
5. M. Concheiro, H. E. Jones, R. E. Johnson, R. Choo and M. A. Huestis, Preliminary buprenorphine sublingual tablet pharmacokinetic data in plasma, oral fluid, and sweat during treatment of opioid-dependent pregnant women. Ther Drug Monit. 33 (5):619-626. (2011).
6. W. Bosker and M. Huestis. Oral fluid testing for drugs of abuse. Clin Chem. 55(11): 1910-1931. (2009).
7. J. Couto, L. Webster, M. Romney et al., Use of an algorithm applied to urine drug screening to assess adherence to an OxyContin® regimen. Journal of Opioid Management, 5, 359-364 (2009).
8. J. Couto, L. Webster, M. Romney et al., Use of an algorithm applied to urine drug screening to assess adherence to a hydrocodone regimen. Journal of Clinical Pharm and Ther, 26, 200-207 (2011).
9. E. Parzen, On Estimation of a Probability Density Function and Mode. Ann. Math. Statist. 33:3, 1065--1076 (1962).
10. Substance Abuse and Mental Health Services Administration. Clinical drug testing in primary care. Technical Assistance Publication (TAP) 32. HHS Publication No. (SMA) 12-4668. Rockville, Md.: Substance Abuse and Mental Health Services Administration.(2012).
11. H. Gjerde, J. Mordal, A. S. Christophersen, J. G. Bramness and J. Morland. Comparison of drug concentrations in blood and oral fluid collected with the intercept® sampling sevice. J Anal Toxicol, 34: 204-209. (2010).
12. S. W. Toennes, S. Steinmeyer, H. J. Maurer, M. R. Moeller and G. F Kauert. Screening for drugs of abuse in oral fluid—correlation of analysis results with serum in forensic cases. J Anal Toxicol. 29: 22-27. (2005).
13. L. R. Webster, The role of urine drug testing in chronic pain management: 2013 update. Pain Medicine News Special ed. 45-50. (2013).
14.A. R. Absalom, V. Mani, T. DeSmet et al., Pharmacokinetic models for propofol—defining and illuminating the devil in the detail. Br J Anaesth 103:26-37 (2009).
15. R. D. Mosteller, Simplified calculation of body surface area. N Engl J Med. 317(17): 1098. (1987).
16. R. O. Gilcher, Apheresis: principles and practices. In: Rossi E C, Simon T L, Moss G S, Gould S A, ed. Principles of transfusion medicine, 2nd ed. Baltimore: Williams and Wilkins. P. 537-546. (1996).
17. B. E. Cole. Recognizing and preventing medication diversion. Fam Pract Manag. 8(9): 37-41. (2001).

1. A method of determining a risk a subject is non-compliant with a prescribed drug regimen, wherein the subject is of a particular age, weight, and gender and has been prescribed a daily dose of the drug, the method comprising:
determining a concentration of a primary metabolite of the drug in an oral fluid sample of the subject;
determining a normalized metabolite concentration as a function of at least the concentration of the primary metabolite, the age, the weight, the height and the gender of the subject;
comparing the normalized metabolite concentration to normalized metabolite concentrations from a control population to provide a metabolite concentration variance; and
determining the risk the subject is non-compliant as a function of at least the metabolite concentration variance.

2. The method of claim 1 further comprising determining a calculated blood volume associated with the subject, wherein the normalized metabolite concentration is determined as a function of at least the calculated blood volume.

3. The method of claim 1, wherein the normalized metabolite concentration is determined as a function of at least the prescribed daily dose of the drug.

4. The method of claim 1, wherein the normalized metabolite concentration is determined as a function of at least an adjustment factor associated with the drug.

5. The method of claim 1, wherein the normalized metabolite concentration is determined as a function of a lean body weight associated with the subject.

6. The method of claim 1, wherein the normalized metabolite concentration is determined as a function of a body surface area associated with the subject.

7. The method of claim 1, wherein the normalized metabolite concentration is determined as a function of a logarithmic transformation of at least some combination of the prescribed daily dose of the drug, the age, the weight, the height and the gender associated with the subject.

8. The method of claim 1, wherein the normalized metabolite concentration is determined as a function of a calculated blood volume, an adjustment factor associated with the drug, and a logarithmic transformation of the concentration of the primary metabolite of the drug in the oral fluid, a lean body weight, a body surface area associated with the subject, and the prescribed daily dose of the drug.

9. The method of claim 1, wherein the normalized metabolite concentration is determined as a function of a calculated blood volume, an adjustment factor associated with the drug, and a logarithmic transformation of the concentration of the primary metabolite of the drug in the oral fluid, a lean body weight associated with the subject, and the prescribed daily dose of the drug.

10. The method of claim 1, wherein the normalized metabolite concentration is determined as a function of a calculated blood volume, an adjustment factor associated with the drug, and a logarithmic transformation of the concentration of the primary metabolite of the drug in the oral fluid, a lean body weight, and a body surface area associated with the subject.

11. The method claim 7, wherein the logarithmic transformation is a natural logarithmic transformation.

12.-14. (canceled)

15. The method of claim 1, wherein the normalized metabolite concentrations from a control population represent a Gaussian distribution.

16. The method of claim 15, wherein the Gaussian distribution includes about 95% of the subject population within +/−2 standard deviations.

17. The method of claim 15, wherein the Gaussian distribution includes about 68% of the subject population within +/−1 standard deviation.

18. The method of claim 1 wherein the drug is selected from the group consisting of controlled-release oxycodone, oxycodone, controlled release morphine, morphine, extended release morphine, hydrocodone, methadone, and a combination of controlled-release oxycodone and oxycodone.

19. (canceled)

20. The method of claim 1 wherein the drug comprises buprenorphine, benzodiazepine, a benzodiazepine metabolite, marijuana, an antidepressant, an anticonvulsant, an amphetamine derivative, an attention deficit hyperactivity disorder (ADHD) drug, an Autism spectrum disorder (ASD) drug, methylphenidate, dexamphetamine, lisdexamphetamine, amphetamine, an opioid or an antipsychotic drug.

21.-31. (canceled)

32. A method of generating a compliance report associated with a subject, the method comprising:
determining a prescribed daily dose of a drug associated with the subject;
determining an age, a weight, and a gender associated with the subject;
estimating a blood volume associated with the subject;
obtaining an oral fluid sample associated with the subject;
determining a concentration of a primary metabolite of the drug in the oral fluid of the subject;
submitting the primary metabolite concentration to a rules engine to produce a rules engine output that describes a relationship between the primary metabolite concentration and the prescribed daily dose of the drug; and
generating a compliance report comprising the rules engine output.

33.-65 (canceled)

66. A system for generating a compliance report associated with a subject, the system comprising:
an input device to receive a drug metabolite concentration, a prescribed daily dose of a drug, an age, a weight, and a gender associated with the subject;
a memory for storing a normalization rule and the prescribed daily dose of the drug, the age, the weight, and the gender associated with the subject;
a processor to:
estimate a blood volume associated with the subject,
normalize the drug metabolite concentration based on the normalization rule, and
generate a compliance report that describes a relationship between the drug metabolite concentration and the prescribed daily dose of the drug; and
an output device to display the compliance report.

67.-99. (canceled)

100. A computer readable medium storing instructions structured to cause a computing device to:
receive a drug metabolite concentration, a prescribed daily dose of a drug, an age, a weight, and a gender associated with the subject;
store a normalization rule and the prescribed daily dose of the drug, the age, the weight, and the gender associated with the subject;
estimate a blood volume associated with the subject;
normalize the drug metabolite concentration based on the normalization rule;
generate a compliance report that describes a relationship between the drug metabolite concentration and the prescribed daily dose of the drug; and
display the compliance report.

101.-167. (Canceled)
